(19) United States
(12) Patent Application Publication
(10) Pub. No.: US 2023/0112484 A1
(43) Pub. Date: Apr. 13, 2023

(54) COMPOSITIONS FOR CROP PROTECTION

(71) Applicant: Agrematch Ltd., Rehovot (IL)

(72) Inventors: Ifat SHUB, RaAnana (IL); Yael PHILLIP, Ness Ziona (IL); Yosef GEVA, Hatzor Ashdod (IL)

(73) Assignee: Agrematch Ltd., Rehovot (IL)

(21) Appl. No.: 17/793,991

(22) PCT Filed: Jan. 20, 2021
(86) PCT No.: PCT/IL2021/050062

§ 371 Date: Jul. 20, 2022

Related U.S. Application Data
(60) Provisional application No. 63/029,873, filed on May 26, 2020.
(60) Provisional application No. 62/963,510, filed on Jan. 20, 2020.

(51) Int. Cl. A01N 37/20; A01N 43/10; A01N 43/40; A01N 43/78; A01P 13/00; A01P 21/00; C07C 233/27

(52) U.S. Cl. CPC A01N 37/20; A01N 43/10; A01N 43/40; A01N 43/78; A01P 13/00; A01P 21/00; C07C 233/27

(57) ABSTRACT

The present invention provides compounds and herbicidal compositions comprising thereof. Further, use of herbicidal compositions or kit for controlling plant growth is also provided.

### CROSS REFERENCE TO RELATED APPLICATIONS

This application claims the benefit of priority under 35 U.S.C. § 119(e) of U.S. Provisional Patent Application Nos. 62/963,510, filed on Jan. 20, 2020 and 63/029,873 filed on May 26, 2020. The contents of the above applications are all incorporated by reference as if fully set forth herein in their entirety.

### FIELD OF THE INVENTION

The present invention relates to agricultural compositions and methods of using thereof.

### BACKGROUND OF THE INVENTION

The protection of crops from undesirable vegetation, which inhibits crop growth, is a constantly recurring problem in agriculture. Many different herbicides have been used over the years to control and kill such weeds. However, problems have more recently been experienced with the use of many of these herbicides, as the weeds have developed a significant resistance against them over time. In some locations the conventional herbicides are therefore no longer sufficiently effective. There is, therefore, an ongoing demand for development of new herbicides.

### SUMMARY OF THE INVENTION

In one aspect of the invention, there is provided a herbicidal composition comprising an effective amount of a compound, or a salt thereof, wherein the compound is represented by Formula IIIc:

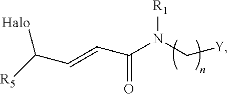

wherein: Y represents an isopropyl group, (C₂-C₁₀) alkyl group, a substituted (C₂-C₁₀) alkyl group, an alkyne group, a substituted alkyne group, a (C₃-C₁₀) cycloalkyl group, a substituted (C₃-C₁₀) cycloalkyl, a heteroaryl, a substituted heteroaryl, a heterocyclyl, a substituted heterocyclyl, an aliphatic ring, a bicyclic aliphatic ring, or a combination thereof; R₁ is selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an alkylhydroxy group, (C₁-C₅ alkyl)-R₂—(C₁-C₅ alkyl)₀₋₂, an alkoxy group, an amino group, a guanidine group, a thioalkoxy group, a thioalkyl group, a hydroxy group, a mercapto group, an allyl group, a vinyl group, a cyano group, a nitro group, an alkylamino group, an alkylamide group, a keto group, a halo group, a haloalkyl group, an azo group and a carboxylic acid derivative; R₅ is selected from the group consisting of: a linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a halo group, a C₁-C₇ alkoxy group, an amino group, a thioalkoxy group, a thioalkyl group, a hydroxy group, a mercapto group, an allyl group, C₁-C₇ ether group, a vinyl group, a cyano group, a nitro group, a C₁-C₇ alkylamino group and a C₁-C₇ alkylamide group or any combination thereof; and n represents an integer in a range from 0 to 10.

In some embodiments, the compound is represented by Formula IIId:

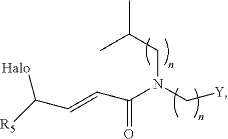

wherein each n independently represents an integer in a range from 0 to 5.

In some embodiments, the Y represents an isopropyl group, (C₂-C₁₀) alkyl group, a substituted (C₂-C₁₀) alkyl group, an alkyne group, a substituted alkyne group, a trifluoromethyl group, a pyran, a thiopyran, a pyridine, a substituted pyran, a substituted thiopyran, a substituted pyridine, a thiophene, a substituted thiophene, a thiazole, a substituted thiazole, an imidazole, a substituted imidazole, a furan, a substituted furan, or a combination thereof.

In some embodiments, the R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

In some embodiments, the compound is represented by Formula IVc:

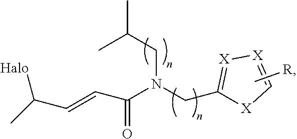

### or Formula IVd:

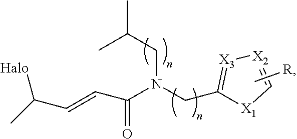

wherein: X represents a heteroatom selected from N, S and O; X₁, X₂, and X₃ are independently selected from the group consisting of: C, CH, S, O, N, and NH; and R represents a substituent, independently selected from the group consisting of: a halo group, an alkyl group, hydrogen, an alkoxy group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, the compound is represented by Formula Vc:

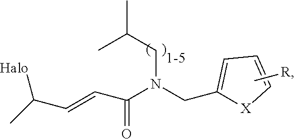

### or Formula Vd:

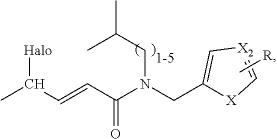

wherein X, X₁, and X₂ each independently represent a heteroatom selected from N, S and O.

In some embodiments, the compound is represented by Formula VIIa:

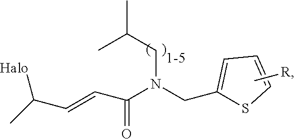

### or Formula VIIb:

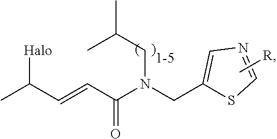

In some embodiments, the R represents a substituent selected from the group consisting of: a halo group, a linear C₁-C₅ alkyl group, a branched C₁-C₅ alkyl group, a C₁-C₅ alkoxy group, a C₁-C₅ ether, and a hydroxy group or any combination thereof.

In some embodiments, the compound is selected from the group consisting of:

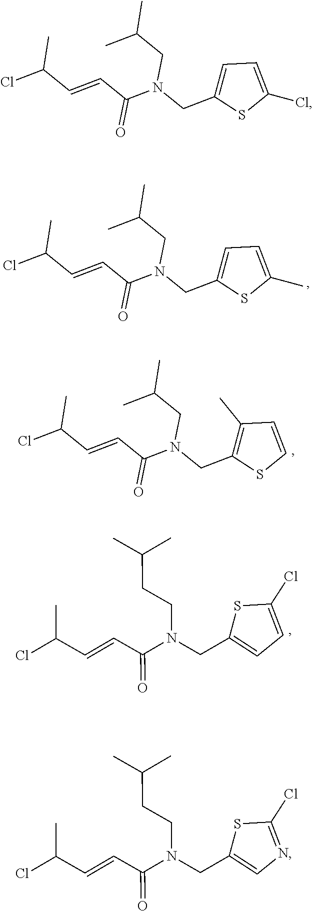 [SMILES: CC(Cl)/C=C/C(=O)N(Cc1ccc(Cl)s1)CC(C)C ; Cc1ccc(CN(CC(C)C)C(=O)/C=C/C(C)Cl)s1 ; Cc1ccsc1CN(CC(C)C)C(=O)/C=C/C(C)Cl ; CC(Cl)/C=C/C(=O)N(CCC(C)C)Cc1ccc(Cl)s1 ; CC(Cl)/C=C/C(=O)N(CCC(C)C)Cc1cnc(Cl)s1]

In some embodiments, the compound is represented by Formula IXc:

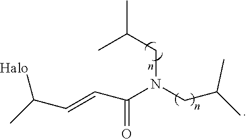

In some embodiments, the compound is selected from the group consisting of:

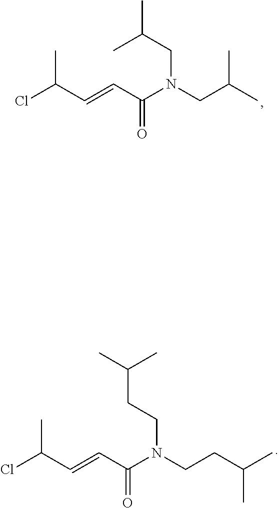 [SMILES: CC(Cl)/C=C/C(=O)N(CC(C)C)CC(C)C ; CC(Cl)/C=C/C(=O)N(CCC(C)C)CCC(C)C]

In some embodiments, the compound is represented by Formula IXd:

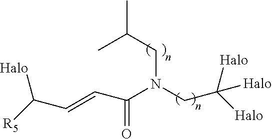

In some embodiments, the compound is:

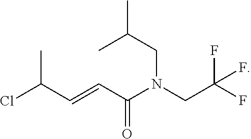 [SMILES: CC(Cl)/C=C/C(=O)N(CC(C)C)CC(F)(F)F]

In some embodiments, the compound is represented by Formula XIc:

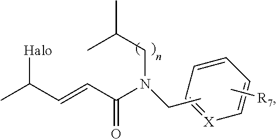

wherein: X is N; n represents an integer in a range from 0 to 5; and R₇ represents a substituent, selected from the group consisting of: a halo group, an alkyl group, an alkoxy group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is represented by Formula XIIc:

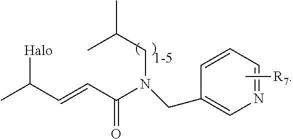

In some embodiments, the compound is selected from the group consisting of:

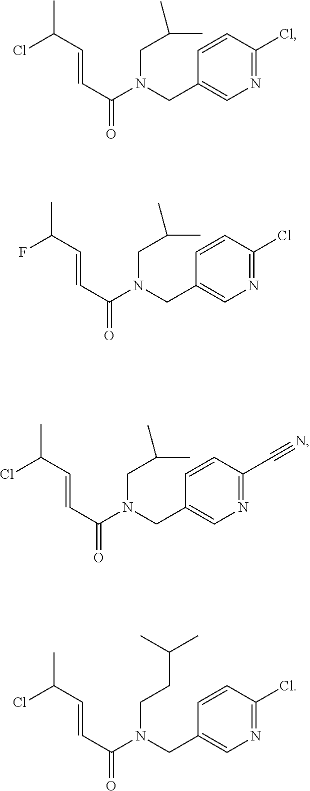 [SMILES: CC(Cl)/C=C/C(=O)N(Cc1ccc(Cl)nc1)CC(C)C ; CC(F)/C=C/C(=O)N(Cc1ccc(Cl)nc1)CC(C)C ; CC(Cl)/C=C/C(=O)N(Cc1ccc(C#N)nc1)CC(C)C ; CC(Cl)/C=C/C(=O)N(CCC(C)C)Cc1ccc(Cl)nc1]

In some embodiments, the compound is represented by Formula XVa:

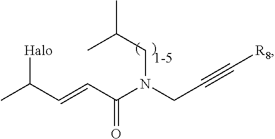

wherein: R₈ represents a substituent, selected from the group consisting of: methyl group, isopropyl group, an alkylhydroxy group, an alkoxy group, a halo group, an alkyl group, a substituted alkyl group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is selected from the group consisting of:

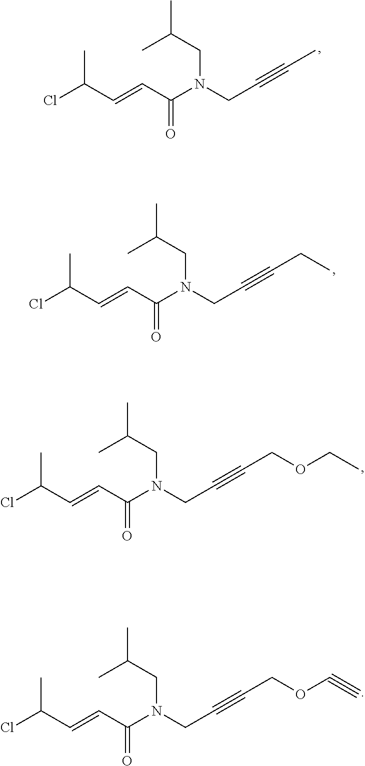 [SMILES: CC#CCN(CC(C)C)C(=O)/C=C/C(C)Cl ; CCC#CCN(CC(C)C)C(=O)/C=C/C(C)Cl ; CCOCC#CCN(CC(C)C)C(=O)/C=C/C(C)Cl ; C#COCC#CCN(CC(C)C)C(=O)/C=C/C(C)Cl]

In another aspect, there is provided a herbicidal composition comprising an effective amount of the compound of the present invention.

In some embodiments, the herbicidal composition comprises an additive selected from a solvent, a surfactant, an emulsifier, or any combination thereof.

In some embodiments, the surfactant is a non-ionic surfactant.

In some embodiments, the emulsifier is a non-ionic emulsifier or an anionic emulsifier.

In some embodiments, the herbicidal composition comprises 5% to 40% weight per weight (w/w) of the compound.

In some embodiments, the herbicidal composition comprises 0.01% to 20% weight per weight (w/w) of the additive.

In some embodiments, the herbicidal composition comprises at least two additives at a w/w ratio ranging from 1:1 to 1:0.05.

In some embodiments, a w/w ratio of the compound to the additive is between 3:1 and 1:10.

In some embodiments, the herbicidal composition further comprises an agriculturally acceptable carrier.

In another aspect, there is provided a compound, or a salt thereof, wherein the compound is represented by Formula IIIc:

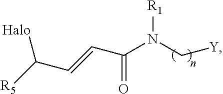

wherein: Y represents an isopropyl group, (C₂-C₁₀) alkyl group, a substituted (C₂-C₁₀) alkyl group, an alkyne group, a substituted alkyne group, a (C₃-C₁₀) cycloalkyl group, a substituted (C₃-C₁₀) cycloalkyl an heteroaryl, a substituted heteroaryl, an heterocyclyl, a substituted heterocyclyl, a bicyclic aromatic ring, an aliphatic ring, an unsaturated aliphatic ring, a bicyclic aliphatic ring, or a combination thereof; R₁ is selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an alkylhydroxy group, (C₁-C₅ alkyl)-R₂—(C₁-C₅ alkyl)₀₋₂, an alkoxy group, an amino group, a guanidine group, a thioalkoxy group, a thioalkyl group, a hydroxy group, a mercapto group, an allyl group, a vinyl group, a cyano group, a nitro group, an alkylamino group, an alkylamide group, a keto group, a halo group, a haloalkyl group, an azo group and a carboxylic acid derivative; R₅ is selected from the group consisting of: a linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a halo group, a C₁-C₇ alkoxy group, an amino group, a thioalkoxy group, a thioalkyl group, a hydroxy group, a mercapto group, an allyl group, C₁-C₇ ether group, a vinyl group, a cyano group, a nitro group, a C₁-C₇ alkylamino group and a C₁-C₇ alkylamide group or any combination thereof; and n represents an integer in a range from 0 to 10.

In some embodiments, the compound is represented by Formula IIId:

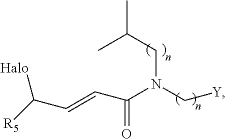

wherein each n independently represents an integer in a range from 0 to 5.

In some embodiments, the Y represents an isopropyl group, (C₂-C₁₀) alkyl group, a substituted (C₂-C₁₀) alkyl group, an alkyne group, a substituted alkyne group, a trifluoromethyl group, a pyran, a thiopyran, a pyridine, a substituted pyran, a substituted thiopyran, a substituted pyridine, a thiophene, a substituted thiophene, a thiazole, a substituted tiazole, an imidazole, a substituted imidazole, or a combination thereof.

In some embodiments, the R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

In some embodiments, the compound is represented by Formula IVc:

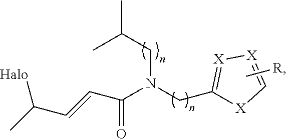

### or Formula IVd:

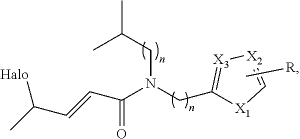

wherein: X represents a heteroatom; X₁, X₂, and X₃ are independently selected from the group consisting of: C, CH, S, O, N, and NH; and R represents a substituent, independently selected from the group consisting of: a halo group, an alkyl group, hydrogen, an alkoxy group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, the compound is represented by Formula Vc:

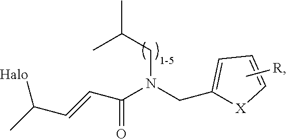

### or Formula Vd:

wherein X, X₁, and X₂ each independently represent a heteroatom.

In some embodiments, the compound is represented by Formula VIIa:

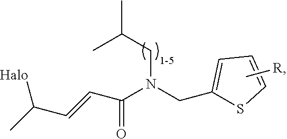

### or Formula VIIb:

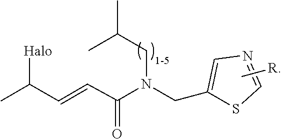

In some embodiments, the R represents a substituent selected from the group consisting of: a halo group, a linear C₁-C₅ alkyl group, a branched C₁-C₅ alkyl group, a C₁-C₅ alkoxy group, a C₁-C₅ ether, and a hydroxy group or any combination thereof.

In some embodiments, the compound is selected from the group consisting of:

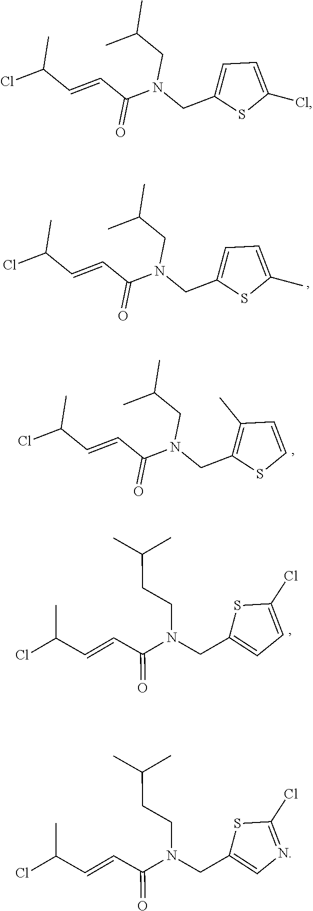 [SMILES: CC(Cl)/C=C/C(=O)N(Cc1ccc(Cl)s1)CC(C)C ; Cc1ccc(CN(CC(C)C)C(=O)/C=C/C(C)Cl)s1 ; Cc1ccsc1CN(CC(C)C)C(=O)/C=C/C(C)Cl ; CC(Cl)/C=C/C(=O)N(CCC(C)C)Cc1ccc(Cl)s1 ; CC(Cl)/C=C/C(=O)N(CCC(C)C)Cc1cnc(Cl)s1]

In some embodiments, the compound is represented by Formula IXc:

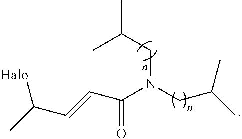

In some embodiments, the compound is selected from the group consisting of:

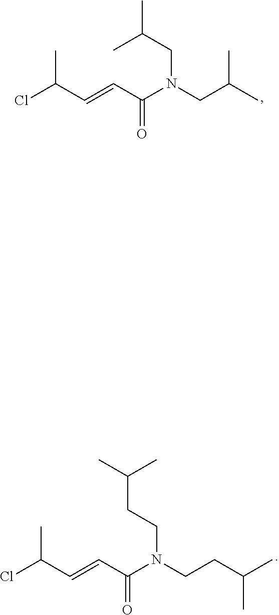 [SMILES: CC(Cl)/C=C/C(=O)N(CC(C)C)CC(C)C ; CC(Cl)/C=C/C(=O)N(CCC(C)C)CCC(C)C]

In some embodiments, the compound is represented by Formula XIc:

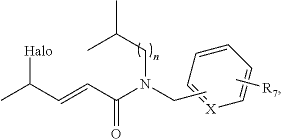

wherein: X represents a heteroatom; n represents an integer in a range from 0 to 5; and R₇ represents a substituent, selected from the group consisting of: a halo group, an alkyl group, an alkoxy group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is represented by Formula XIIc:

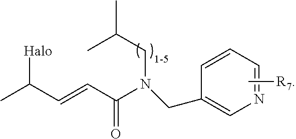

In some embodiments, the compound is selected from the group consisting of:

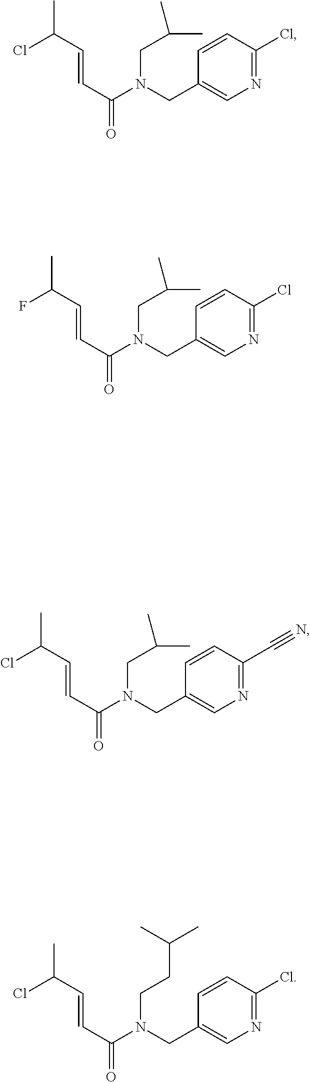 [SMILES: CC(Cl)/C=C/C(=O)N(Cc1ccc(Cl)nc1)CC(C)C ; CC(F)/C=C/C(=O)N(Cc1ccc(Cl)nc1)CC(C)C ; CC(Cl)/C=C/C(=O)N(Cc1ccc(C#N)nc1)CC(C)C ; CC(Cl)/C=C/C(=O)N(CCC(C)C)Cc1ccc(Cl)nc1]

In some embodiments, the compound is represented by Formula XVa:

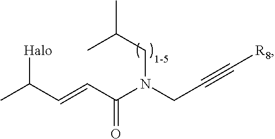

wherein: R₈ represents a substituent, selected from the group consisting of: methyl group, isopropyl group, an alkylhydroxy group, an alkoxy group, a halo group, an alkyl group, a substituted alkyl group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is selected from the group consisting of

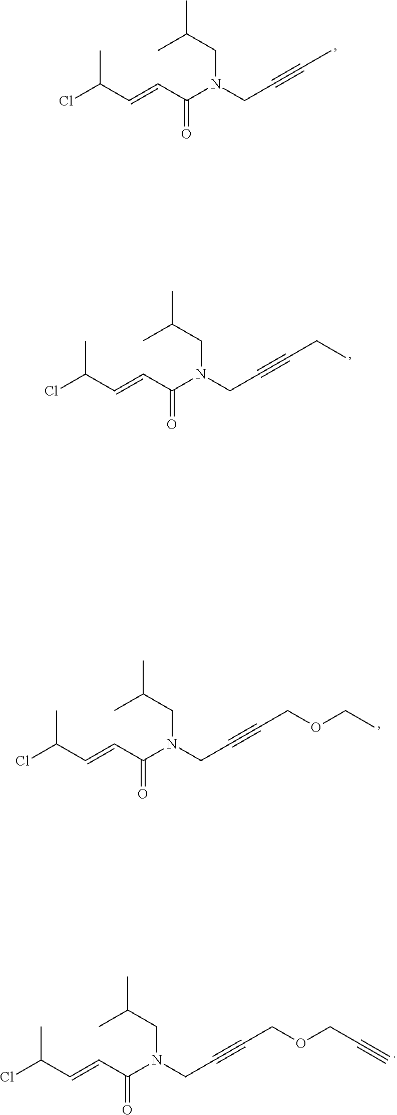 [SMILES: CC#CCN(CC(C)C)C(=O)/C=C/C(C)Cl ; CCC#CCN(CC(C)C)C(=O)/C=C/C(C)Cl ; CCOCC#CCN(CC(C)C)C(=O)/C=C/C(C)Cl ; C#CCOCC#CCN(CC(C)C)C(=O)/C=C/C(C)Cl]

In another aspect, there is provided a method for controlling plant growth, comprising applying to the plant, a part of the plant, a seed of the plant, or the area under cultivation, an effective amount of the herbicidal composition of the present invention, or the compound of the present invention.

In some embodiments, the plant is selected from a crop plant and a weed.

In some embodiments, the effective amount is between 0.5 grams per hectare (g/ha) and 1500 g/ha.

In some embodiments, the applying comprises pre-emergence, post-emergence, pre-planting the crop plant, post-planting the crop plant, including any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 represents the chemical structures of compounds 1-6; and of phenyl-based derivatives;

FIGS. 2A-2B present the result of the systemic/contact experiment on Amaranthus palmeri plants; and

FIGS. 3A-C present the chemical structures of compounds 7-18.

### DETAILED DESCRIPTION OF THE INVENTION

The present invention is directed to a compound and to an herbicidal composition comprising the compound of the invention and use thereof such as for herbicidal use.

The present invention is also directed to a method for preventing or reducing undesired plant growth, comprising contacting a compound or a herbicidal composition described herein with a plant, a part of the plant (e.g. seed) or an area under cultivation.

Compounds disclosed by the present invention, were discovered by methods of computational screening using big-data analysis platforms. After several optimization cycles in silico, a chemical library containing several molecules was obtained. The disclosed compounds were selected based on results obtained from plant growth experiments (e.g. germination and foliar experiments), as represented in Tables 1 and 2.

Compounds, which induced at least 70% inhibition of Amaranthus palmeri germination in soil germination experiment (see Example section), were selected as suitable herbicidal candidates.

The present invention is based, in part, on the finding that alkyl derivatives and substituted pyridine derivatives exhibit an herbicidal effect (e.g. against Amaranthus palmeri) upon applying to a plant, or the area under cultivation.

The present invention is based, in part, on the finding that substituted thiophene derivatives exhibit an herbicidal effect (e.g. against Amaranthus palmeri and Lolium rigidum) upon applying to a plant, or the area under cultivation.

The present invention is based, in part, on the finding that substituted thiazole derivatives exhibit an herbicidal effect (e.g. against Amaranthus palmeri) upon applying to a plant, or the area under cultivation.

The present invention is based, in part, on the finding that alkyne derivatives, and substituted alkyne derivatives exhibit an herbicidal effect (e.g. against Amaranthus palmeri) upon applying to a plant, or the area under cultivation.

In contrary to the above, it was found that some structurally related phenyl-based derivatives were substantially less potent (e.g. pre-emergency of Amaranthus palmeri) as compared to heteroaryl derivates and to alkyl derivatives.

Thus, the present invention provides a compound or a composition for use in controlling or preventing undesired plant growth.

Furthermore, some of the compounds of the invention didn't show an herbicidal effect against field crops of the genus Zea (e.g. Zea mays), and genus Triticum (e.g. Triticum vulgare) and at the same time exhibited selective herbicidal activity against different weed species, as represented in the Examples section.

### Compounds and Compositions

According to an aspect of the present invention, there is provided a compound, or a salt thereof, wherein the compound is represented by Formula I:

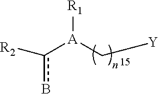

wherein: Y represents a methyl group, isopropyl group, (C₂-C₁₀) alkyl group, a substituted (C₂-C₁₀) alkyl group, an alkyne group, a substituted alkyne group, a (C₃-C₁₀) cycloalkyl group, a substituted (C₃-C₁₀) cycloalkyl an heteroaryl, a substituted heteroaryl, an heterocyclyl, a substituted heterocyclyl, a bicyclic aromatic ring, an aliphatic (C₃-C₁₀) cycloalkyl, an unsaturated aliphatic ring, a bicyclic aliphatic ring, or a combination thereof;
B is selected from the group consisting of NH, NH₂, S, SH, O, OH, CH₂ and CH;
A is selected from N, and CH; [symbol]
represents a single bond or a double bond; n represents an integer in a range from 0 to 10; R₁ represents a substituent, independently selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an alkylhydroxy group, (C₁-C₅ alkyl)-R₂—(C₁-C₅ alkyl)₀₋₂, an alkoxy group, an amino group, a guanidine group, a thioalkoxy group, a thioalkyl group, a hydroxy group, a mercapto group, an allyl group, a vinyl group, a cyano group, a nitro group, an alkylamino group, an alkylamide group, a keto group, a halo group, a haloalkyl group, an azo group and a carboxylic acid derivative or any combination thereof; and R₂ is selected from the group consisting of: (C₁-C₁₀) alkyl group, a substituted (C₂-C₁₀) alkyl group, (C₁-C₁₀) alkenyl group, a substituted (C₂-C₁₀) alkenyl group, a (C₃-C₁₀) cycloalkyl group, an alkoxy group, an ether, a carboxylic acid derivative, N, S, O, P, phosphonate, phosphate, thiophosphate, —S(O)—, —S(O)₂—, —OS(O)₂—, and —S(O)₂N— or any combination thereof; or R₁ and R₂ are interconnected so as to form a 4 to 8-membered ring.
In some embodiments, n represents an integer in a range from 0 to 10, from 0 to 1, from 1 to 3, from 3 to 5, from 5 to 7, from 7 to 10, including any range between.

In some embodiments, R₂ comprises an aliphatic (C₁-C₁₀) alkyl chain. In some embodiments, R₂ comprises an aliphatic (C₁-C₁₀) alkyl chain, wherein the chain is optionally branched. In some embodiments, R₂ comprises an aliphatic (C₁-C₁₀) alkyl chain, wherein the chain comprises an unsaturated bond. In some embodiments, R₂ comprises an aliphatic (C₁-C₁₀) alkyl chain, wherein the chain comprises a substituent as described hereinbelow. In some embodiments, R₂ is devoid of an aromatic substituent.

In some embodiments, Y is a heteroaromatic ring. In some embodiments, Y is a fused ring or polycylic C6-C12 aromatic and/or aliphatic ring comprising at least one heteroatom.

In some embodiments, Y is a 6-membered heteroaromatic ring comprises one or two heteroatoms. In some embodiments, Y is selected from pyran, thiopyran, pyridazine, pyrimidine, and pyrazine including a derivative (such as substituted pyran, thiopyran, pyridazine, pyrimidine, and pyrazine) or a combination thereof. In some embodiments, Y is devoid of phenyl.

In some embodiments, Y is pyridine or a substituted pyridine.

In some embodiments, Y is a heteroaromatic ring. In some embodiments, Y is a 5-membered heteroaromatic ring comprising one, two or three heteroatoms. In some embodiments, Y is selected from thiophene, oxazole, isoxazole, isothiazole, thiazole, oxothiolane, furan, 1,3-dioxolane, pyrazole, imidazole, triazole, oxadiazole, or a combination thereof.

In some embodiments, Y is a thiazole or a substituted thiazole. In some embodiments, Y is a thiophene, or a substituted thiophene. In some embodiments, Y is devoid of furan. In some embodiments, Y is devoid of pyrrole.

In some embodiments, the compound, or salt thereof is represented by Formula II:

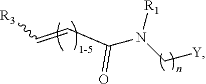

wherein [symbol] represents cis- or trans-configuration of the double bond; R₃ represents a substituent, independently comprising or being selected from the group consisting of: a C₁-C₇ alkyl group, a branched C₁-C₇ haloalkyl group, a C₁-C₇ haloalkyl group, a C₁-C₇ alkylhydroxy group, a C₁-C₇ alkoxy group, an amino group, a thioalkoxy group, an ether, a thioalkyl group, a hydroxy group, a mercapto group, an allyl group, a vinyl group, a cyano group, a nitro group, a C₁-C₇ alkylamino group, a cyclic C₁-C₇ alkyl group, and a C₁-C₇ alkylamide group or any combination thereof.

In some embodiments, the compound is represented by Formula III:

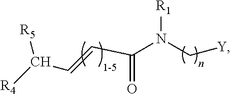

wherein R₄ and R₅ are independently selected from the group consisting of a linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a halo group, a C₁-C₇ alkoxy group, an amino group, a thioalkoxy group, a thioalkyl group, a hydroxy group, a mercapto group, an allyl group, C₁-C₇ ether group, a vinyl group, a cyano group, a nitro group, a C₁-C₇ alkylamino group and a C₁-C₇ alkylamide group or any combination thereof.

In some embodiments, the compound is represented by Formula IIIa:

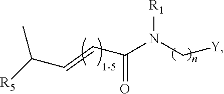

### or Formula IIIb:

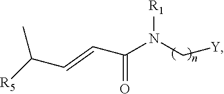

wherein R₅ is selected from the group consisting of a linear C₁-C₇ alkyl group, a branched C₁-C₇ alkyl group a branched C₁-C₇ haloalkyl group, a linear C₁-C₇ haloalkyl group, a halo group, a C₁-C₇ alkoxy group, an amino group, or any combination thereof.

In some embodiments, the compound is represented by Formula IIIc:

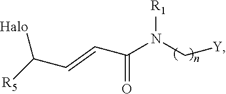

wherein R₁, R₅, Y and n are as described herein above.

In some embodiments, R₁ represents a substituent, independently selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an alkylhydroxy group, (C₂-C₁₀alkyl)-R′₂—(C₂-C₁₀alkyl)₀₋₂, (C₂-C₁₀alkyl)-X—(C₂-C₁₀alkyl)₀₋₂, C₁-C₆ alkyl, C₁-C₁₀ haloalkyl, C₁-C₁₀ alkyl-NR′₂, C₁-C₁₀ alkyl-OR′, C₁-C₁₀ alkyl-CONR′₂, C₁-C₁₀ alkyl-COOR′, C₁-C₁₀ alkyl-CNNR′₂, C₁-C₁₀ alkyl-CSNR′₂, C₁-C₁₀alkyl-SR′, —CONH(C₁-C₆ alkyl), —CON(C₁-C₆ alkyl)₂, —CO₂H, —CO₂R′, —COR′, —OCOR′, —CONH₂, —CONR′₂, —CNNR′₂, —CSNR′₂, —CONH—OH, —CONH—NH₂, or any combination thereof, wherein R1 is devoid of phenyl and/or benzyl, and wherein each R′ independently represents hydrogen, or is selected from the group comprising optionally substituted C₁-C₁₀ alkyl, optionally substituted C₃-C₁₀ cycloalkyl, and wherein X is O, N, or S.

In some embodiments, the compound is represented by Formula IIIc1:

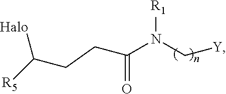

wherein R₁, R₅, Y and n are as described herein above.

In some embodiments, the compound is represented by Formula IIId:

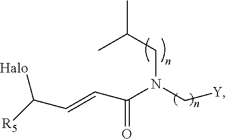

wherein each n independently represents an integer in a range from 0 to 5, and R₅ and Y are as described herein above.

In some embodiments, Y represents an isopropyl group, (C₂-C₁₀) alkyl group, a substituted (C₂-C₁₀) alkyl group, an alkyne group, a substituted alkyne group, a pyran, a thiopyran, a pyridine, a substituted pyran, a substituted thiopyran, a substituted pyridine, a thiophene, a substituted thiophene, a thiazole, a substituted thiazole, an imidazole, a substituted imidazole, or a combination thereof.

In some embodiments, Y represents a methyl group, isopropyl group, (C₂-C₁₀) alkyl group, an optionally substituted (C₂-C₁₀) alkyne group, an optionally substituted (C₂-C₁₀) alkene group, a substituted (C₂-C₁₀) alkyl group, a (C₃-C₁₀) cycloalkyl group, an alkyne group, a substituted alkyne group, an C₁-C₁₀ alkylhydroxy group, (C₂-C₁₀alkyl)-X—(C₂-C₁₀alkyl)₀₋₂, C₁-C₆ alkyl, C₁-C₁₀ haloalkyl, C₁-C₁₀alkyl-NR′₂, C₁-C₁₀alkyl-OR′, C₁-C₁₀alkyl-CONR′₂, C₁-C₁₀alkyl-COOR′, C₁-C₁₀alkyl-CNNR′₂, C₁-C₁₀alkyl-CSNR′₂, C₁-C₁₀alkyl-SR′, or any combination thereof, wherein X is O, N, or S, and wherein alkyl comprises saturated alkyl or comprises at least one unsaturated bond (double or triple bond).

In some embodiments, R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

In some embodiments, the compound is represented by Formula IV:

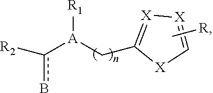

### Formula IVa:

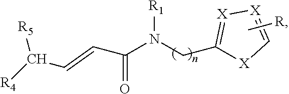

### or Formula IVb:

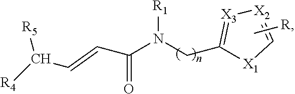

wherein X represents a heteroatom each independently selected from N, S, and O; X₁, X₂, and X₃ are independently selected from the group consisting of: C, CH, S, O, N, and NH; and R represents a substituent, independently selected from the group consisting of: a halo group, an alkyl group, hydrogen, an alkoxy group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, the compound is represented by Formula IVc:

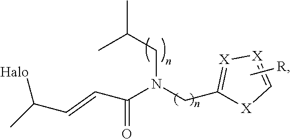

### or Formula IVd:

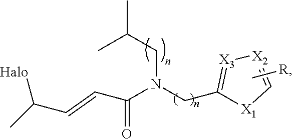

wherein: X represents a heteroatom; X₁, X₂, and X₃ are independently selected from the group consisting of: C, CH, S, O, N, and NH; and R represents a substituent, independently selected from the group consisting of: a halo group, an alkyl group, hydrogen, an alkoxy group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, the compound is represented by Formula V:

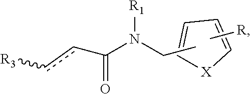

wherein X, R, R₃ and R₁ are as described herein.

In some embodiments, the compound is represented by Formula Va:

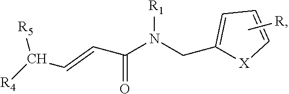

### or Formula Vb:

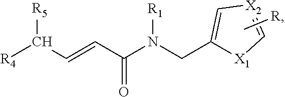

Vc:

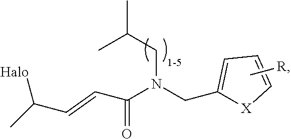

### or Formula Vd:

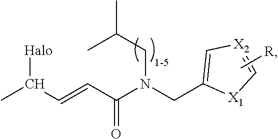

wherein X, X₁, and X₂ each independently represent a heteroatom.

In some embodiments, R represents a substituent, independently selected from the group consisting of: a halo group, an alkyl group, hydrogen, an alkoxy group, a hydroxy group, a cyano group, an ether, and an amino group, or any combination thereof.

In some embodiments, R represents a substituent, independently selected from the group consisting of: a halo group, a C₁-C₇ alkyl group, hydrogen, a C₁-C₇ alkoxy group, a hydroxy group, a mercapto group, a cyano group, a C₁-C₇ ether group or any combination thereof.

In some embodiments, R represents a substituent, independently selected from the group consisting of: a halo group, a C₁-C₇ alkyl group, a C₁-C₇ alkoxy group, a hydroxy group, hydrogen, a hydroxy group, a C₁-C₇ ether group or any combination thereof.

In some embodiments, X is S, O or NH.

In some embodiments, the compound is represented by Formula Vai:

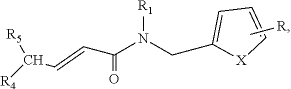

### or by Formula Vaii:

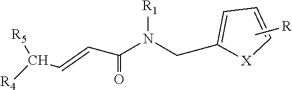

wherein X, R, and R₁ are as described herein. In some embodiments, X is S, or NH. In some embodiments, X is S.

In some embodiments, R represents a substituent selected from the group consisting of: a halo group, a linear C₁-C₅ alkyl group, a branched C₁-C₅ alkyl group, a C₁-C₅ alkoxy group, a C₁-C₅ ether, and a hydroxy group or any combination thereof.

In some embodiments, R₄ and R₅ are independently selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof. In some embodiments, R₄ comprises a halo group. In some embodiments, R₅ is selected from a linear C₁-C₅ alkyl group, or a branched C₁-C₅ alkyl group.

In some embodiments, the compound is represented by Formula Vci:

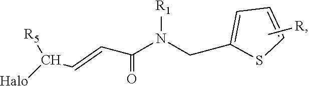

wherein R₅, R₁ and R are as described herein.

In some embodiments, the compound is represented by Formula Vdi:

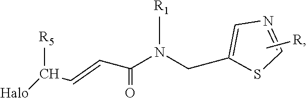

wherein R₅, R₁ and R are as described herein.

In some embodiments, R₁ represents a substituent, independently selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an C₁-C₁₀alkylhydroxy group, (C₂-C₁₀alkyl)-R′₂—(C₂-C₁₀alkyl)₀₋₂, C₁-C₆ alkyl, C₁-C₁₀ haloalkyl, C₁-C₁₀ alkyl-NR′₂, C₁-C₁₀alkyl-OR′, C₁-C₁₀alkyl-CONR′₂, C₁-C₁₀ alkyl-COOR′, C₁-C₁₀ alkyl-CNNR′₂, C₁-C₁₀alkyl-CSNR′₂, C₁-C₁₀alkyl-SR′, or any combination thereof

In some embodiments, the compound is represented by Formula VIa:

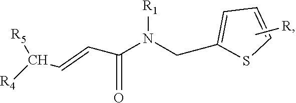

### or Formula VIb:

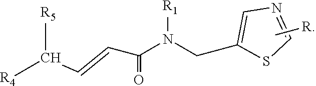

wherein R₅, R₄, R₁ and R are as described herein.

In some embodiments, R represents a substituent selected from the group consisting of: a halo group, a linear C₁-C₅ alkyl group, a branched C₁-C₅ alkyl group, a C₁-C₅ alkoxy group, a C₁-C₅ ether, and a hydroxy group or any combination thereof.

In some embodiments, the compound is represented by Formula VII:

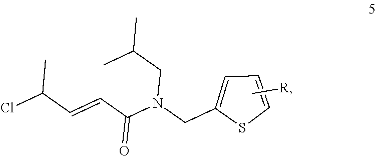

Formula VIIa:

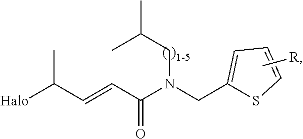

### or Formula VIIb:

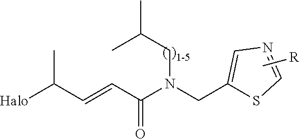

wherein R is are as described herein.

In some embodiments, R represents a substituent selected from the group consisting of: a halo group, a linear C₁-C₅ alkyl group, a C₁-C₅ alkoxy group, and a hydroxy group or any combination thereof.

In some embodiments, the compound is selected from the group consisting of:

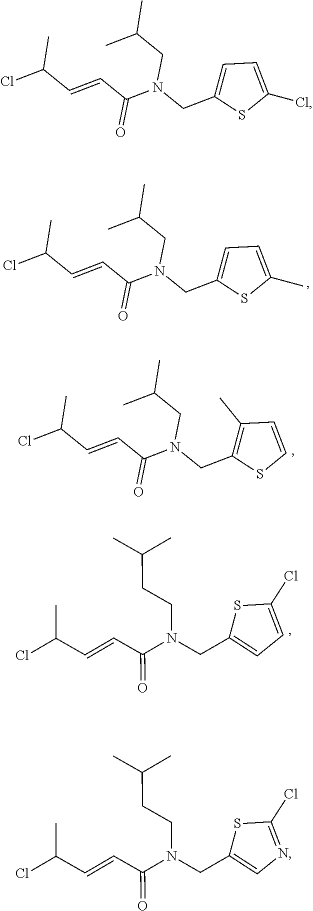 [SMILES: CC(Cl)/C=C/C(=O)N(Cc1ccc(Cl)s1)CC(C)C ; Cc1ccc(CN(CC(C)C)C(=O)/C=C/C(C)Cl)s1 ; Cc1ccsc1CN(CC(C)C)C(=O)/C=C/C(C)Cl ; CC(Cl)/C=C/C(=O)N(CCC(C)C)Cc1ccc(Cl)s1 ; CC(Cl)/C=C/C(=O)N(CCC(C)C)Cc1cnc(Cl)s1]

In some embodiments, the compound is selected from the group consisting of:

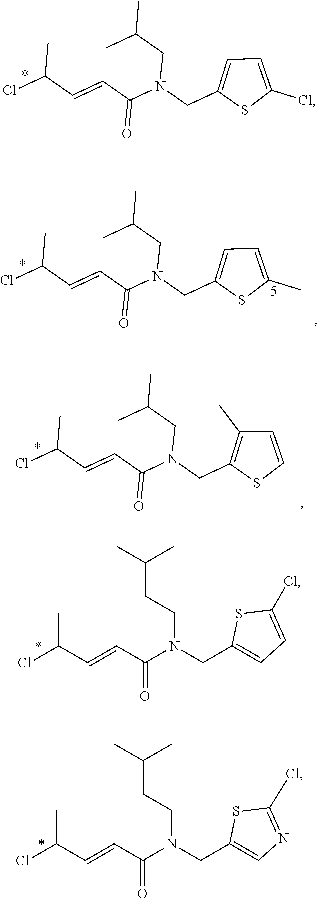

wherein * is referred to any of R or S enantiomers or a mixture thereof.

In some embodiments, the compound is represented by Formula VIII:

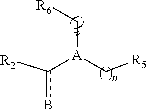

Or Formula VIIIa:

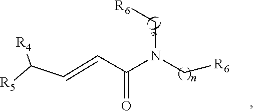

wherein R₂ and B are as described herein, and wherein each R₆ independently represents a methyl group, isopropyl group, (C₂-C₁₀) alkyl group, a substituted (C₂-C₁₀) alkyl group, a (C₃-C₁₀), or a combination thereof; and each n independently represents an integer in a range from 0 to 5.

In some embodiments, the compound is represented by Formula IX:

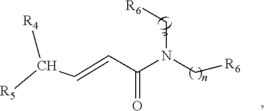

### or Formula IXa:

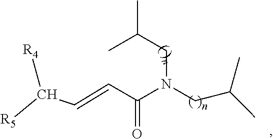

### or Formula IXb:

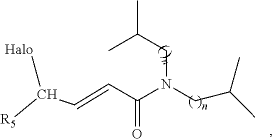

### or Formula IXc:

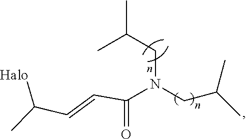

wherein R₄, R₅, R₆ and n are as described herein.

In some embodiments, the compound is represented by Formula Xa:

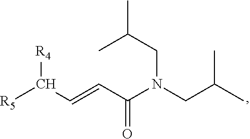

### or Formula Xb:

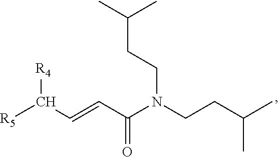

wherein R₄ and R₅ are as described herein.

In some embodiments, the compound is represented by Formula Xc:

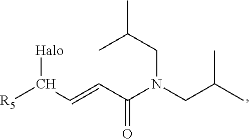

### or Formula Xd:

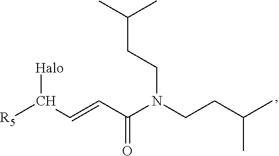

wherein R₅ is as described herein.

In some embodiments, the compound is selected from the group consisting of:

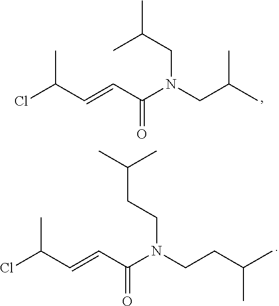

In some embodiments, the compound is selected from the group consisting of:

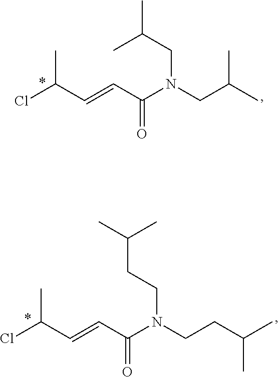 [SMILES: CC(Cl)/C=C/C(=O)N(CC(C)C)CC(C)C ; CC(Cl)/C=C/C(=O)N(CCC(C)C)CCC(C)C]

wherein * is referred to any of R or S enantiomers or a mixture thereof.

In some embodiments, the compound is:

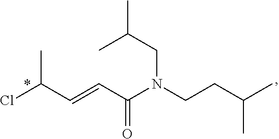 [SMILES: CC(Cl)/C=C/C(=O)N(CCC(C)C)CC(C)C]

wherein * is referred to any of R or S enantiomers or a mixture thereof.

In some embodiments, the compound is represented by Formula IXd:

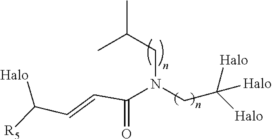

In some embodiments, the compound is:

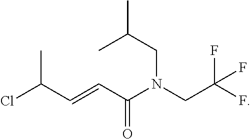 [SMILES: CC(Cl)/C=C/C(=O)N(CC(C)C)CC(F)(F)F]

In some embodiments, the compound is represented by Formula XI:

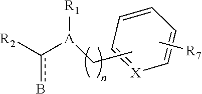

wherein X represents a heteroatom; R₁ and R₂ are as described hereinabove; and R₇ represents a substituent, selected from the group consisting of: a halo group, an alkyl group, an alkoxy group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is represented by Formula XIa:

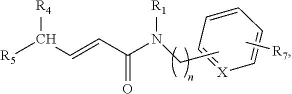

wherein X represents a heteroatom, n represents an integer in a range from 0 to 5, and R₇ represents a substituent, selected from the group consisting of: a halo group, an alkyl group, an alkoxy group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is represented by Formula XIb:

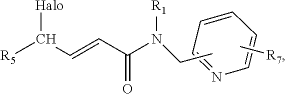

### or by Formula XIc:

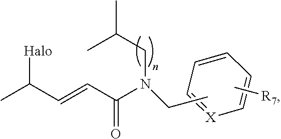

wherein R₁, R₅ and R₇ are as described hereinabove.

In some embodiments, the compound is represented by Formula XId:

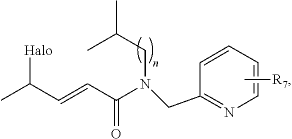

wherein R₇ is as described hereinabove.

In some embodiments, the compound is:

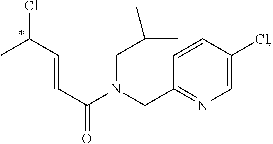 [SMILES: CC(Cl)/C=C/C(=O)N(Cc1ccc(Cl)cn1)CC(C)C]

wherein * is referred to any of R or S enantiomers or a mixture thereof.

In some embodiments, the compound is represented by Formula XII:

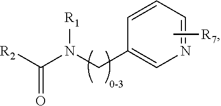

wherein R₇, R₁ and R₂ are as described hereinabove.

In some embodiments, the compound is represented by Formula XIIa:

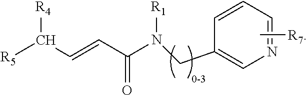

wherein R₇, R₁, R₄ and R₅ are as described hereinabove.

In some embodiments, the compound is represented by Formula XIIb:

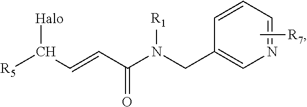

wherein R₁, R₅, and R₇ are as described hereinabove.

In some embodiments, the compound is represented by Formula XIIc:

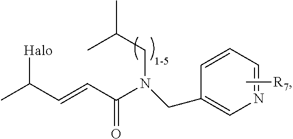

wherein R₇ are as described hereinabove.

In some embodiments, the compound is represented by Formula XIII:

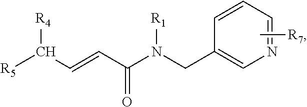

wherein R₅, R₇, R₁ and R₄ are as described hereinabove.

In some embodiments, the compound is represented by Formula XIIIa:

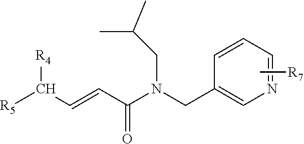

wherein R₅, R₇ and R₄ are as described hereinabove.

In some embodiments, the compound is re resented by Formula XIIIb:

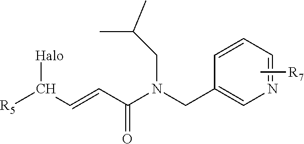

wherein R₅ and R₇ are as described hereinabove. In some embodiments, each R₇ is independently selected from halo, cyano and C₁-C₄ alkyl. In some embodiments, R₇ is devoid of hydrogen.

In some embodiments, the compound is represented by Formula XIIIc:

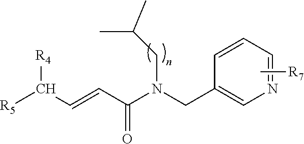

wherein R₅, R₇ and R₄ are as described hereinabove, and n represents an integer in a range from 0 to 10.

In some embodiments, the compound is represented by Formula XIIId:

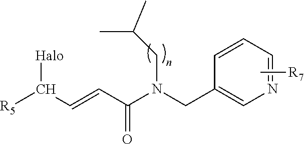

wherein R₅ and R₇ are as described hereinabove, and n represents an integer in a range from 0 to 10. In some embodiments, each R₇ is independently selected from halo, cyano and C1-C4 alkyl. In some embodiments, R₇ is devoid of hydrogen.

In some embodiments, the compound is any of

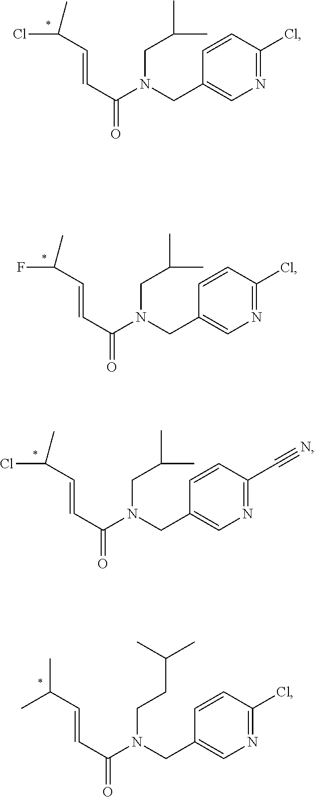 [SMILES: CC(Cl)/C=C/C(=O)N(Cc1ccc(Cl)nc1)CC(C)C ; CC(F)/C=C/C(=O)N(Cc1ccc(Cl)nc1)CC(C)C ; CC(Cl)/C=C/C(=O)N(Cc1ccc(C#N)nc1)CC(C)C ; CC(C)/C=C/C(=O)N(CCC(C)C)Cc1ccc(Cl)nc1]

wherein * is referred to any of R or S enantiomers or a mixture thereof.

In some embodiments, the compound is represented by Formula XIV:

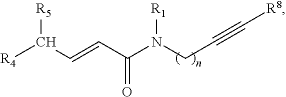

Wherein R₁, R₄ and R₅ are as described hereinabove, and R₈ represents a substituent, selected from the group consisting of: methyl group, isopropyl group, an alkylhydroxy group, an alkoxy group, a halo group, an alkyl group, a substituted alkyl group, an alkyne group, an amino group, a guanidine group, a thioalkoxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

In some embodiments, the compound is represented by Formula XV:

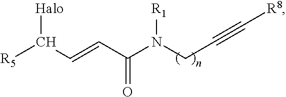

Wherein R₁, R₅ and R₈ are as described hereinabove.

In some embodiments, the compound is represented by Formula XVa:

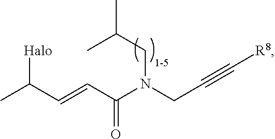

Wherein R₈ are as described hereinabove.

In some embodiments, the compound is selected from the group consisting of:

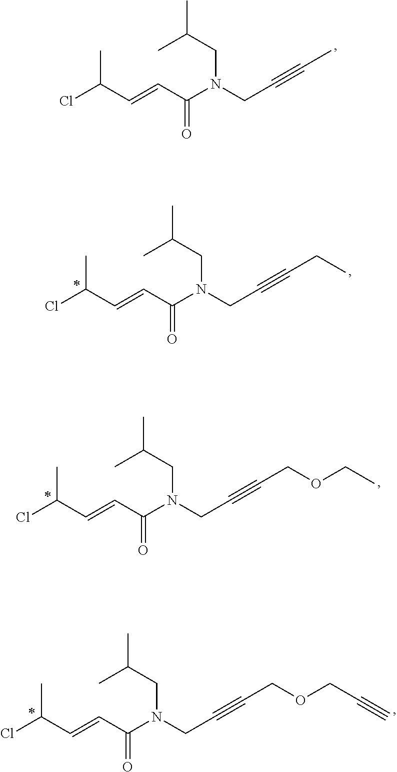 [SMILES: CC#CCN(CC(C)C)C(=O)/C=C/C(C)Cl ; CCC#CCN(CC(C)C)C(=O)/C=C/C(C)Cl ; CCOCC#CCN(CC(C)C)C(=O)/C=C/C(C)Cl ; C#CCOCC#CCN(CC(C)C)C(=O)/C=C/C(C)Cl]

wherein * is referred to any of R or S enantiomers or a mixture thereof.

As used herein the term “C₁-C₇ alkyl” including any C₁-C₇ alkyl related compounds, is referred to any linear or branched alkyl chain comprising between 1 and 6, between 1 and 2, between 2 and 3, between 3 and 4, between 4 and 5, between 5 and 6, between 6 and 7 carbon atoms, including any range therebetween. In some embodiments, C₁-C₇ alkyl comprises any of methyl, ethyl, propyl, butyl, pentyl, iso-pentyl, hexyl, and tert-butyl or any combination thereof. In some embodiments, C₁-C₆ alkyl as described herein further comprises an unsaturated bond, wherein the unsaturated bond is located at 1^{st}, 2^{nd}, 3^{rd}, 4^{th}, 5^{th}, or 6^{th} position of the C₁-C₇ alkyl.

As used herein the term “C₁-C₁₀ alkyl” including any C₁-C₁₀ alkyl related compounds, is referred to any linear or branched alkyl chain comprising between 1 and 10, between 1 and 2, between 2 and 3, between 3 and 4, between 4 and 5, between 5 and 6, between 6 and 8, between 8 and 10 carbon atoms, including any range therebetween. In some embodiments, C₁-C₁₀ alkyl comprises any of methyl, ethyl, propyl, butyl, pentyl, iso-pentyl, hexyl, and tert-butyl or any combination thereof. In some embodiments, C₁-C₁₀ alkyl as described herein further comprises an unsaturated bond, wherein the unsaturated bond is located at 1^{st}, 2^{nd}, 3^{rd}, 4^{th}, 5^{th}, or 6^{th} position of the C₁-C₁₀ alkyl.

As used herein the term “C₁-C₆ alkyl” including any C₁-C₆ alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C₁-C₆ alkyl comprises any of methyl, ethyl, propyl, butyl, pentyl, iso-pentyl, hexyl, and tert-butyl or any combination thereof. In some embodiments, C₁-C₆ alkyl as described herein further comprises an unsaturated bond, wherein the unsaturated bond is located at 1^{st}, 2^{nd}, 3^{rd}, 4^{th}, 5^{th}, or 6^{th} position of the C₁-C₆ alkyl.

As used herein the term “C₁-C₅ alkyl” including any C₁-C₅ alkyl related compounds, is referred to any linear or branched alkyl chain comprising between 1 and 6, between 1 and 2, between 2 and 3, between 3 and 4, between 4 and 5, carbon atoms, including any range therebetween. In some embodiments, C₁-C₅ alkyl comprises any of methyl, ethyl, propyl, butyl, pentyl, iso-pentyl, hexyl, and tert-butyl or any combination thereof. In some embodiments, C₁-C₅ alkyl as described herein further comprises an unsaturated bond, wherein the unsaturated bond is located at 1^{st}, 2^{nd}, 3^{rd}, 4^{th}, 5^{th}, or 6^{th} position of the C₁-C₅ alkyl.

As used herein the term “C₂-C₁₀ alkyl” including any C₂-C₁₀ alkyl related compounds, is referred to any linear or branched alkyl chain comprising between 2 and 10 between 2 and 3, between 3 and 4, between 4 and 5, between 5 and 6, between 6 and 8, between 8 and 10, carbon atoms, including any range therebetween. In some embodiments, C₂-C₁₀ alkyl comprises any of methyl, ethyl, propyl, butyl, pentyl, iso-pentyl, hexyl, octyl nonyl, decyl and tert-butyl or any combination thereof. In some embodiments, C₂-C₁₀ alkyl as described herein further comprises an unsaturated bond, wherein the unsaturated bond is located at 1^{st}, 2^{nd}, 3^{rd}, 4^{th}, 5^{th}, or 6^{th} position of the C₂-C₁₀ alkyl.

As used herein the term “C₃-C₁₀ cycloalkyl” is referred to an optionally substituted C3, C4, C5, C6, C7, C8, C9 or C10 ring. In some embodiments, (C₃-C₁₀) ring comprises optionally substituted cyclopropane, cyclobutene, cyclopentane, cyclohexane, or cycloheptane.

As used herein the term “fused ring” refers to any bicyclic ring (e.g. fused ring, spirocyclic ring, biaryl ring). As used herein the term “fused ring” is referred to a bicyclic (C₆-C₁₂) ring comprising at least one heteroatom, wherein (C₆-C₁₂) ring is as described herein. In some embodiments the terms “bicyclic aromatic ring”, “bicyclic aliphatic ring” are referred to (C₆-C₁₂) rings.

As used herein the term “(C₆-C₁₂) ring” is referred to an optionally substituted C6, C7, C8, C9, C10, C11, or C12 ring. In some embodiments, (C₆-C₁₂) ring is referred to a bicyclic ring (e.g. fused ring, spirocyclic ring, biaryl ring).

As used herein the term “substituted” refers to one or more (e.g. 2, 3, 4, 5, or 6) substituent groups, which is independently selected from (C₀-C₆)alkyl-aryl, (C₀-C₆)alkyl-heteroaryl, (C₀-C₆)alkyl-(C₃-C₅) cycloalkyl, optionally substituted C₃-C₈ heterocyclyl, halogen, —NO₂, —CN, —OH, —CONH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, depending on the substituted group and its position in the molecule.

In some embodiments, the compound or the composition of the invention comprises a derivative of any of the compounds described hereinabove. In some embodiments, the derivative comprises a structural isomer. In some embodiments, the structural isomer comprises any of a conformer, a stereoisomer, and a diastereoisomer, or a combination thereof. In some embodiments, the configuration of the double bond is either trans or cis.

In some embodiments, the compound of the invention comprises a mixture of isomers, wherein the isomers are as described hereinabove. In some embodiments, the compound of the invention is in a form of a racemic mixture. In some embodiments, the compound of the invention comprises one or more compounds having at least 85%, at least 90%, at least 95%, at least 97%, at least 99% enantiomeric purity.

In some embodiments, the composition of the invention comprises a mixture of isomers, wherein the isomers are as described hereinabove. In some embodiments, the composition of the invention is in a form of a racemic mixture. In some embodiments, the composition of the invention comprises one or more compounds having at least 85%, at least 90%, at least 95%, at least 97%, at least 99% enantiomeric purity.

In some embodiments, the composition of the invention is an herbicidal composition, comprising any compound described hereinabove, and an agriculturally acceptable carrier. In some embodiments, the herbicidal composition comprises an herbicidally effective amount of the compound.

### Method of Treatment

In one aspect, there is a method for controlling or preventing the growth of a plant, comprising applying to the plant, a part of the plant, a seed of the plant, and the area under cultivation an effective amount of a compound or of the herbicidal composition of the invention. In some embodiments, the effective amount comprises herbicidally effective amount.

In some embodiments, the method comprise applying an effective amount of a compound or of the herbicidal composition of the invention to a foliage of the plant (e.g. contact herbicide). In some embodiments, the method comprise systemically applying an effective amount of a compound or of the herbicidal composition of the invention to an area under cultivation (e.g. systemic herbicide). In some embodiments, the effective amount is agriculturally effective amount. In some embodiments, the effective amount of the compound or of the composition (e.g. herbicidal composition) of the invention is sufficient for controlling or preventing growth of a weed, wherein controlling is as described herein.

In some embodiments, there is a method for controlling or preventing the growth of a plant. In some embodiments, the method is for controlling or preventing germination of seeds. In some embodiments, the method is for substantially destroying a plant or a part of the plant. In some embodiments, the plant is a crop. In some embodiments, the plant is a broad-leaved plant. In some embodiments, the plant is a weed.

In some embodiments, the herbicidal composition is applied to a plant or to the area under cultivation for accomplishing a total vegetative control. In some embodiments, the herbicidal composition is applied to a plant or to the area under cultivation for accomplishing a complete eradication of a weed.

In some embodiments, the method is for controlling or preventing the growth of monocotyledonous (monocot) weeds and of dicotyledonous (dicot) weeds. In some embodiments, the method is for selectively controlling or preventing the growth of monocotyledonous (monocot) weeds or of dicotyledonous (dicot) weeds.

In some embodiments, there is a method for controlling or preventing the growth of a weed by applying an effective amount of a compound or of the herbicidal composition of the invention to the plant, parts of the plant, seed of the plant, or the area under cultivation.

The term ‘herbicide’ as used herein denotes a compound which controls or modifies the growth of plants. The term ‘herbicidally effective amount’ indicates the quantity of such a compound or combination of such compounds which can control or modify the growth of plants. The term “control or modify” include all deviation from natural development, such as: killing, retardation, leaf burn, albinism, dwarfing, germination prevention and the like. For example, plants that are not killed are often stunted and non-competitive with flowering disrupted. The term ‘plants’ refers to all physical parts of a plant, including seeds, seedlings, saplings, roots, tubers, stems, stalks, foliage, fruits, plants emerging from vegetative propagules, immature vegetation, and established vegetation.

The herbicidal composition of the present invention can be used against any plants growing in an undesired location. For example, the composition of the invention may be used against a large number of agronomically important weeds, including, but not limited to, monocotyledonous (monocot) weeds and dicotyledonous (dicot) weeds. In some embodiments, the composition of the invention is effective against both monocotyledonous weeds and dicotyledonous weeds.

Non-limiting examples of Dicotyledon weeds are: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Emex, Datura, Viola, Galeopsis, Papaver, Trifolium, Abutilon, and Centaurea.

Non-limiting examples of Monocotyledon weeds are: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbris tylis, Sagittaria, Eleocharis, Scirpus, Paspalum, Ischaenum, Sphenoclea, Dactyloctenium, Agrostis, Alopecurus and Apera.

As defined herein, the term “weeds” includes undesirable crop species such as volunteer crops. For example, a crop species growing in an area where a different crop is being cultivated may be considered a ‘volunteer’.

As defined herein, the term “area under cultivation” is intended to include fields, hard landscapes such as driveways, paths, patios, roads, pavements, railways and the like, as well as soil, or established vegetation.

In some embodiments, the herbicidal composition is applied, depending on the concentration, for controlling weeds in field crops such as: Zea, Oryza, Triticum, Hordeum, Avena, Secale, Sorghum, Panicum, Solanum, Ananas, Asparagus, Fagopyrum, Phaseolus, and Allium.

In some embodiments, the herbicidal composition is applied for selective weed control in field crops.

In some embodiments, the herbicidal composition is applied, depending on the concentration, for controlling weeds in perennial cultures such as: decorative tree plantings, fruit orchards, vineyards, citrus groves, nut orchards, banana plantations, coffee plantations, tea plantations, rubber plantations, oil palm plantations, cocoa plantations, soft fruit plantings and hop fields, and for the selective combating of weeds in annual cultures.

In some embodiments, the herbicidal composition is applied to a plant for growth regulation.

In some embodiments, the herbicidal composition is applied to a plant for accomplishing a total vegetative control.

In some embodiments, there is a method for controlling the growth of undesirable vegetation by pre-emergence or post-emergence application of the composition. In one embodiment, therefore, the herbicidal composition of the invention is applied as a pre-emergent application. In a further embodiment, the herbicidal composition of the invention is applied as a post-emergent application.

In some embodiments, applying the herbicidal composition of the invention can significantly reduce the levels (e.g., amount, concentration) of a herbicide when applied together with the herbicidal composition of the invention.

In some embodiments, the compounds of the present invention will exert a dual and possibly synergistic herbicidal activity in combination with any another herbicidal active substance.

In some embodiments, the method comprises applying the herbicidal composition of the invention at an effective amount. In some embodiments, the effective amount is between 0.5 grams per hectare (g/ha) and 1500 g/ha, between 5 g/ha and 1000 g/ha, between 5 g/ha and 500 g/ha, between 5 g/ha and 150 g/ha, 10 grams per hectare (g/ha) and 1500 g/ha, between 10 g/ha and 1000 g/ha, between 10 g/ha and 500 g/ha, between 10 g/ha and 150 g/ha, 20 grams per hectare (g/ha) and 1500 g/ha, between 20 g/ha and 1000 g/ha, between 20 g/ha and 500 g/ha, between 20 g/ha and 150 g/ha, between 40 g/ha and 1500 g/ha, between 45 g/ha and 1500 g/ha, between 50 g/ha and 1500 g/ha, between 60 g/ha and 1500 g/ha, between 80 g/ha and 1500 g/ha, between 100 g/ha and 1500 g/ha, between 120 g/ha and 1500 g/ha, between 150 g/ha and 1500 g/ha, between 45 g/ha and 1300 g/ha, between 50 g/ha and 1300 g/ha, between 60 g/ha and 1300 g/ha, between 80 g/ha and 1300 g/ha, between 100 g/ha and 1300 g/ha, between 120 g/ha and 1300 g/ha, between 150 g/ha and 1300 g/ha, between 45 g/ha and 1200 g/ha, between 50 g/ha and 1200 g/ha, between 60 g/ha and 1200 g/ha, between 80 g/ha and 1200 g/ha, between 100 g/ha and 1200 g/ha, between 120 g/ha and 1200 g/ha, or between 150 g/ha and 1200 g/ha, including any range therebetween. Each possibility represents a separate embodiment of the invention.

In some embodiments, the method comprises applying the herbicidal composition between 150 g/ha and 1200 g/ha, as shown in the results presented in Example 6 and Example 7.

In some embodiments, the method comprises applying the herbicidal composition of the invention at an effective dose from 1 to 500 g/ha, from 1 to 5 g/ha, from 5 to 10 g/ha, from 10 to 20 g/ha, from 2 to 5 g/ha, from 50 to 60 g/ha, from 60 to 70 g/ha, from 70 to 90 g/ha, from 90 to 100 g/ha, from 100 to 150 g/ha, from 150 to 200 g/ha, from 200 to 300 g/ha, from 300 to 400 g/ha, from 400 to 500 g/ha, including any range or value therebetween, wherein the effective dose is sufficient for controlling weeds. Each possibility represents a separate embodiment of the invention.

In some embodiments, the effective dose of the compound for controlling weeds is at least 0.5 g/ha, at least 1 g/ha, at least 2 g/ha, at least 5 g/ha, at least 10 g/ha, at least 50 g/ha, at least 100 g/ha, at least 150 g/ha, at least 200 g/ha, at least 300 g/ha, at least 400 g/ha, at least 400 g/ha, at least 500 g/ha, at least 600 g/ha, at least 700 g/ha, at least 800 g/ha, at least 1000 g/ha, at least 1500 g/ha, at least 1800 g/ha, at least 2000 g/ha, at least 2200 g/ha, at least 2400 g/ha, at least 2600 g/ha, at least 2800 g/ha, at least 3000 g/ha, including any range or value therebetween. Each possibility represents a separate embodiment of the invention.

As used herein, the term “controlling” in the context of herbicides, indicates that the growth rate of weeds is essentially nullified or is reduced by at least 20%, at least 30%, at least 40%, at least 50%, at least 60%, at least 70%, at least 80%, at least 90%, including any value therebetween, of the growth of weeds in a comparable situation lacking the presence of the disclosed compounds or a composition of matter containing same.

It will be recognized that these embodiments are susceptible to various modifications and alternative forms well known to those of skill in the art.

### Herbicidal Composition

According to an aspect of the invention, there is provided a composition comprising one or more compounds described herein, and an additive selected from a solvent, a surfactant, an emulsifier, or any combination thereof. In some embodiments, the composition of the invention comprising an effective amount (e.g. herbicidally effective amount) of one or more compounds described herein, and an additive selected from a solvent, a surfactant, an emulsifier, or any combination thereof. In some embodiments, the additive is an agriculturally acceptable carrier. According to an aspect of the invention, there is provided a formulation comprising one or more compounds described herein, and an additive selected from a solvent, a surfactant, an emulsifier, or any combination thereof.

According to an aspect of the invention, there is provided a kit or a combined preparation comprising (i) a first compartment comprising an effective amount (e.g. herbicidally effective amount) of one or more compounds described herein, and (ii) a second compartment comprising an agriculturally acceptable carrier. In some embodiments, the first compartment further comprises an agriculturally effective amount of any of a fertilizer, a pesticide, or an agriculturally acceptable carrier (solid or liquid carrier). In some embodiments, the ratio between the first compartment and the second compartment is so as to result in a formulation and/or herbicidal composition of the invention, upon mixing thereof.

In some embodiments, there is an agricultural composition comprising an agriculturally effective amount of a compound of the invention. In some embodiments, the agriculturally effective amount of the compound of the invention is sufficient for controlling or preventing growth of a weed, wherein controlling is as described herein.

In some embodiments, the agricultural composition or the herbicidal composition of the invention is for use in controlling or preventing growth of a weed, wherein controlling is as described herein.

In some embodiments, the kit of the invention is for use in controlling or preventing growth of a weed, wherein controlling is as described herein.

In some embodiments, the agricultural composition or the herbicidal composition of the invention comprises a herbicidally effective amount of a compound of the invention being of at least 0.5 g/dunam, at least 1 g/dunam, at least 2 g/dunam, at least 5 g/dunam, at least 10 g/dunam, at least 50 g/dunam, at least 100 g/dunam, at least 150 g/dunam, at least 200 g/dunam, at least 300 g/dunam, at least 400 g/dunam, at least 400 g/dunam, at least 500 g/dunam, at least 600 g/dunam, at least 700 g/dunam, at least 800 g/dunam, at least 1000 g/dunam, at least 1500 g/dunam, at least 1800 g/dunam, at least 2000 g/dunam, at least 2200 g/dunam, at least 2400 g/dunam, at least 2600 g/dunam, at least 2800 g/dunam, at least 3000 g/dunam, including any range or value therebetween. Each possibility represents a separate embodiment of the invention.

In some embodiments, the surfactant is a non-ionic surfactant. In some embodiments, the surfactant comprises is a polymeric non-ionic surfactant. In some embodiments, the surfactant of the invention consists essentially of a non-ionic surfactant, such as a non-ionic polymeric surfactant.

In some embodiments, the emulsifier is a non-ionic emulsifier of an anionic emulsifier.

The term “non-ionic” is well-understood by a skilled artisan, as being referred to compound (e.g. an uncharged molecule, or a neutral polymer) being substantially devoid of a negative and/or positive charge. One skilled in the art will appreciate, that the terms “non-ionic” and “uncharged” are used herein interchangeably.

In some embodiments, the composition or the formulation comprises 5% to 40% weight per weight (w/w), 7% to 40% (w/w), 9% to 40% (w/w), 10% to 40% (w/w), 15% to 40% (w/w), 5% to 30% (w/w), 7% to 30% (w/w), 9% to 30% (w/w), 10% to 30% (w/w), 15% to 30% (w/w), 5% to 20% (w/w), 7% to 20% (w/w), 9% to 20% (w/w), or 10% to 20% (w/w) of the compound, including any range therebetween. Each possibility represents a separate embodiment of the invention.

In some embodiments, the composition or the formulation, comprises between 0.5 mg/ml and 20 mg/ml, 0.7 mg/ml and 20 mg/ml, 1 mg/ml and 20 mg/ml, 5 mg/ml and 20 mg/ml, 0.5 mg/ml and 10 mg/ml, 1 mg/ml and 10 mg/ml, 1 mg/ml and 5 mg/ml mg/ml, 5 mg/ml and 10 mg/ml, or between 10 mg/ml and 20 mg/ml, of the compound, including any range therebetween. Each possibility represents a separate embodiment of the invention.

In some embodiments, any of the composition, the herbicidal composition or the formulation comprises 0.01% to 20% (w/w), 0.04% to 20% (w/w), 0.05% to 20% (w/w), 0.1% to 20% (w/w), 0.5% to 20% (w/w), 1% to 20% (w/w), 5% to 20% (w/w), 0.01% to 15% (w/w), 0.04% to 15% (w/w), 0.05% to 15% (w/w), 0.1% to 15% (w/w), 0.5% to 15% (w/w), 1% to 15% (w/w), 5% to 15% (w/w), 0.01% to 10% (w/w), 0.04% to 10% (w/w), 0.05% to 10% (w/w), 0.1% to 10% (w/w), 0.5% to 10% (w/w), 1% to 10% (w/w), or 5% to 10% (w/w) of the additive, including any range therebetween. Each possibility represents a separate embodiment of the invention. In some embodiments, any of the composition, the herbicidal composition or the formulation comprises between 10 and 90%, between 10 and 30%, between 30 and 50%, between 50 and 70%, between 70 and 90%, of the additive, including any range therebetween. Each possibility represents a separate embodiment of the invention.

In some embodiments, the composition, or the formulation comprises at least two additives at a w/w ratio ranging from 1:1 to 1:0.05, 1:1 to 1:0.08, 1:1 to 1:0.08, 1:1 to 1:0.1, 1:1 to 1:0.5, 1:1 to 1:0.9, 1:0.5 to 1:0.05, 1:0.5 to 1:0.08, 1:0.5 to 1:0.08, 1:0.5 to 1:0.1, or 1:0.5 to 1:0.9, including any range therebetween. Each possibility represents a separate embodiment of the invention.

In some embodiments, water is used as a solvent. In other embodiments, organic solvents are used as solvents. In some embodiments, the composition comprises agriculturally acceptable solvents. Non-limiting examples of agriculturally acceptable solvents include but are not limited to glycol ethers, such as butyl diglycol, N-formyl-morpholine, shorter aliphatic alcohols, propylene carbonate or combination thereof.

Additional non-limiting examples of suitable solvents include but are not limited to: xylene, toluene or alkyl naphthalenes, chlorobenzenes, chloroethylenes, aliphatic hydrocarbons, such as cyclohexane or paraffins, mineral and vegetable oils, alcohols, such as butanol or glycol as well as their ethers and esters (e.g. ethyl lactate), ketones, such as acetone, methyl ethyl ketone, methyl isobutyl ketone or cyclohexanone, strongly polar solvents, such as dimethylformamide (DMF) and dimethylsulfoxide (DMSO), as well as water.

In some embodiments, the composition or the formulation comprises DMSO. In some embodiments, the w/w concentration of DMSO within the composition or formulation is between 0.01 and 50%, between 0.1 and 50%, between 0.01 and 20%, between 0.01 and 10%, between 0.01 and 5%, between 0.1 and 10%, including any range or value therebetween. Each possibility represents a separate embodiment of the invention.

In some embodiments, the composition or the formulation comprises Xylene. In some embodiments, the w/w concentration of DMSO within the composition or formulation is between 0.01 and 50%, between 0.1 and 50%, between 0.01 and 20%, between 0.01 and 10%, between 0.01 and 5%, between 0.1 and 10%, including any range or value therebetween. Each possibility represents a separate embodiment of the invention.

In some embodiments, the w/w concentration of the solvent within the composition or formulation is between 0.01 and 99%, between 0.1 and 99%, between 0.01 and 50%, between 0.01 and 20%, between 0.01 and 10%, between 0.1 and 10%, between 10 and 99%, between 10 and 20%, between 20 and 30%, between 30 and 40%, between 40 and 50%, between 50 and 90%, between 60 and 90%, between 70 and 90%, between 70 and 80%, between 80 and 90%, between 90 and 99%, between 90 and 95%, between 95 and 99%, including any range or value therebetween. Each possibility represents a separate embodiment of the invention.

In some embodiments, the composition or the formulation, comprises Tween20 at a concentration of between 0.01% v/v and 10% v/v, between 0.1% v/v and 1% v/v, between 0.1% v/v and 0.5% v/v, between 0.5% v/v and 1% v/v, including any range therebetween. Each possibility represents a separate embodiment of the invention.

In some embodiments, the composition or the formulation, comprises Tween80 at a concentration of between 0.01% v/v and 10% v/v, between 0.1% v/v and 1% v/v, between 0.1% v/v and 0.5% v/v, between 0.5% v/v and 1% v/v, including any range therebetween. Each possibility represents a separate embodiment of the invention.

In some embodiments, the composition or the formulation comprises Ethoxylated Castor Oil (ECO). In some embodiments, the w/w concentration of ECO within the composition or formulation is between 0.01% v/v and 10% v/v, between 0.1% v/v and 1% v/v, between 0.1% v/v and 0.5% v/v, between 0.5% v/v and 1% v/v, including any range therebetween. Each possibility represents a separate embodiment of the invention.

In some embodiments, the composition or the formulation comprises Linear Alkylbenzene Sulfonate (Ca-Las). In some embodiments, the w/w concentration of Ca-LAS within the composition or formulation is between 0.01% v/v and 10% v/v, between 0.1% v/v and 1% v/v, between 0.1% v/v and 0.5% v/v, between 0.5% v/v and 1% v/v, including any range therebetween. Each possibility represents a separate embodiment of the invention.

In some embodiments, the composition, or the formulation comprises at least two emulsifiers at a w/w ratio ranging from 1:1 to 1:0.05, 1:1 to 1:0.08, 1:1 to 1:0.08, 1:1 to 1:0.1, 1:1 to 1:0.5, 1:1 to 1:0.9, 1:0.5 to 1:0.05, 1:0.5 to 1:0.08, 1:0.5 to 1:0.08, 1:0.5 to 1:0.1, or 1:0.5 to 1:0.9, including any range therebetween. Each possibility represents a separate embodiment of the invention. In some embodiments, the composition, or the formulation comprises ECO and Ca-LAS at a w/w ratio ranging from 1:1 to 1:0.05, 1:1 to 1:0.08, 1:1 to 1:0.08, 1:1 to 1:0.1, 1:1 to 1:0.5, 1:1 to 1:0.9, 1:0.5 to 1:0.05, 1:0.5 to 1:0.08, 1:0.5 to 1:0.08, 1:0.5 to 1:0.1, or 1:0.5 to 1:0.9, including any range therebetween. Each possibility represents a separate embodiment of the invention.

In some embodiments, the composition or the formulation comprises Break-Thru. In some embodiments, the w/w concentration of Break-Thru within the composition or formulation is between 0.01% v/v and 5% v/v, between 0.01% v/v and 1% v/v, between 0.01% v/v and 0.5% v/v, or between 0.05% v/v and 1% v/v, including any range therebetween. Each possibility represents a separate embodiment of the invention.

According to an aspect of the invention, there is provided an herbicidal composition comprising one or more compounds described herein, or the composition described hereinabove, and an agriculturally acceptable carrier. In some embodiments, the herbicidal composition further comprises an agriculturally accepted salt of the compound. In some embodiments, the herbicidal composition comprises an herbicidally effective amount of one or more compounds described herein.

In some embodiments, the herbicidal composition comprises 0.5% to 10% weight per weight (w/w), 0.9% to 10% (w/w), 1% to 10% (w/w), 1.2% to 10% (w/w), 3% to 10% (w/w), 5% to 10% (w/w), 0.5% to 7% (w/w), 0.9% to 7% w/w), 1% to 7% (w/w), 1.2% to 7% (w/w), 3% to 7% (w/w), 0.5% to 5% (w/w), 0.9% to 5% (w/w), 1% to 5% (w/w), or 1.2% to 5% (w/w), of the compound or the composition, including any range therebetween. Each possibility represents a separate embodiment of the invention.

In some embodiments, the herbicidal composition is dilutable up to a concentration of the compound or the composition between 0.5% and 10% (w/w), between 0.7% and 10% (w/w), between 1% and 10% (w/w), between 2% and 10% (w/w), between 0.7% and 7% (w/w), between 1% and 7% (w/w), between 2% and 7% (w/w), between 0.7% and 5% (w/w), between 1% and 5% (w/w), or between 2% and 5% (w/w), within the herbicidal composition, including any range therebetween. Each possibility represents a separate embodiment of the invention.

As used herein “dilutable” refers to capable of or, suitable for being diluted in water or an organic solvent. In some embodiments, “dilutable” refers to suitable for being diluted in agriculturally acceptable solvents as described hereinabove.

In some embodiments, the herbicidal composition or the composition comprises the compound of the invention at a weight per volume (w/v) concentration of 0.01 to 500 mg/ml. In some embodiments, the concentration of the compound within the herbicidal composition or within the composition is from 0.01 to 0.05 mg/ml, from 0.03 to 0.06 mg/ml, from 0.06 to 0.1 mg/ml, from 0.1 to 1 mg/ml, from 0.1 to 0.5 mg/ml, from 0.5 to 1 mg/ml, 0.1 to 10 mg/ml, from 1 to 10 mg/ml, from 1 to 5 mg/ml, from 5 to 10 mg/ml, from 10 to 20 mg/ml, from 20 to 30 mg/ml, from 30 to 40 mg/ml, from 40 to 50 mg/ml, from 50 to 60 mg/ml, from 60 to 70 mg/ml, from 70 to 80 mg/ml, from 80 to 90 mg/ml, from 90 to 100 mg/ml including any range or value therebetween. Each possibility represents a separate embodiment of the invention. In some embodiments, the herbicidal composition or the composition comprises the compound at a concentration between 100 and 200 mg/ml, between 100 and 150 mg/ml, between 150 and 200 mg/ml, between 200 and 300 mg/ml, between 200 and 250 mg/ml, between 250 and 300 mg/ml, between 300 and 500 mg/ml, between 300 and 350 mg/ml, between 350 and 400 mg/ml, between 400 and 450 mg/ml, between 450 and 500 mg/ml including any range or value therebetween. Each possibility represents a separate embodiment of the invention.

In some embodiments, the agricultural carrier is a soil or a plant growth medium. In some embodiments, the agricultural carrier is selected from the group consisting of: a fertilizer, a plant-based oil, and a humectant, or any combination thereof.

In some embodiments, the agricultural carrier is a solid carrier. Non-limiting examples of solid carriers include but are not limited to: mineral carriers (e.g. kaolin clay, pyrophyllite, bentonite, montmorillonite, diatomaceous earth, acid white soil, vermiculite, pearlite, loam, and silica), inorganic salts (e.g. ammonium sulfate, ammonium phosphate, ammonium nitrate, urea, ammonium chloride, and calcium carbonate), alginate, vermiculite, seed cases, other plant and animal products, or any combination thereof including a granule, a pellets, and a suspension.

In some embodiments, the agricultural carrier is a liquid carrier. In some embodiments, the agricultural carrier is an aqueous solution. In some embodiments, the agricultural carrier is an aqueous solution comprising a surfactant. In some embodiments, the agricultural carrier is an aqueous solution comprising an emulsifier. In some embodiments, the agricultural carrier comprises water. Non-limiting examples of liquid carriers include but are not limited to: soybean oil and cottonseed oil, glycerol, ethylene glycol, polyethylene glycol, propylene glycol, polypropylene glycol, or any combination thereof.

In some embodiments, the agricultural carrier comprises a mixture of any one of the aforementioned ingredients, such as pesta (flour and kaolin clay), agar or flour-based pellet in loam, sand, and clay.

In some embodiments, the herbicidal composition comprises a liquid (e.g. an aqueous) formulation. Non-limiting examples of formulations include but are not limited to: emulsions, wettable powders, suspensions, powders, dusts, pastes, soluble powders, granules, suspension-emulsion concentrates, natural and synthetic substances impregnated with active compound, very fine capsules in polymeric substances and in coating compositions for seed, and ULV formulations.

These formulations are produced in known manner, for example by mixing the active compounds with extenders, such as liquid solvents and/or solid carriers, optionally with the use of surface-active agents (e.g. is emulsifying agents, dispersing agents, and foam-forming agents).

In some embodiments, the formulation or the herbicidal composition further comprises an additive. Non-limiting examples of additives including but are not limited to: emuslifiers, sticking agents, spreading agents, surfactants, synergists, penetrants, compatibility agents, buffers, acidifiers, defoaming agents, thickeners, and drift retardants or any combination thereof.

In some embodiments, a w/w concentration of the additive within the formulation or the herbicidal composition is from 0.01% to 10%, from 0.01% to 0.1%, from 0.1% to 0.5%, from 0.5% to 1%, from 0.1% to 0.3%, from 0.3% to 0.5%, from 0.5% to 0.7%, from 0.7% to 1%, from 1% to 1.5%, from 1.5% to 2%, from 2% to 2.5%, from 2.5% to 3%, from 3% to 3.5%, from 3.5% to 4%, from 4% to 5%, from 5% to 6%, from 6% to 7%, from 7% to 8%, from 8% to 10% including any range or value therebetween. Each possibility represents a separate embodiment of the invention.

In some embodiments, the formulation comprises a tackifier or adherent. Such agents are useful for combining the compound of the invention with carriers to yield a coating composition. Such compositions may aid to maintain contact between the compound of the invention or a composition containing thereof, and a weed.

In one embodiment, an adherent is selected from the group consisting of: alginate, a gum, a starch, a lecithin, formononetin, polyvinyl alcohol, alkali formononetinate, hesperetin, polyvinyl acetate, a cephalin, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and a polyoxyethylene-polyoxybutylene block copolymer. Other examples of adherent compositions that can be used in the synthetic preparation include those described in EP 0818135, CA 1229497, WO 2013090628, EP 0192342, WO 2008103422 and CA 1041788.

In some embodiments, water is used as a solvent. In other embodiments, organic solvents are used as auxiliary solvents.

Non-limiting examples of suitable auxiliary solvents include but are not limited to: xylene, toluene or alkyl naphthalenes, chlorobenzenes, chloroethylenes, aliphatic hydrocarbons, such as cyclohexane or paraffins, mineral and vegetable oils, alcohols, such as butanol or glycol as well as their ethers and esters (e.g. ethyl lactate), ketones, such as acetone, methyl ethyl ketone, methyl isobutyl ketone or cyclohexanone, strongly polar solvents, such as dimethylformamide (DMF) and dimethylsulfoxide (DMSO), as well as water.

In some embodiments, the composition comprises DMSO. In some embodiments, the w/w concentration of DMSO within the composition or formulation is between 0.01 and 50%, between 0.1 and 50%, between 0.01 and 20%, between 0.01 and 10%, between 0.01 and 5%, between 0.1 and 10%, including any range or value therebetween. Each possibility represents a separate embodiment of the invention.

In some embodiments, the w/w concentration of the solvent within the composition or formulation is between 0.01 and 99%, between 0.1 and 99%, between 0.01 and 50%, between 0.01 and 20%, between 0.01 and 10%, between 0.1 and 10%, between 10 and 99%, between 10 and 20%, between 20 and 30%, between 30 and 40%, between 40 and 50%, between 50 and 90%, between 60 and 90%, between 70 and 90%, between 70 and 80%, between 80 and 90%, between 90 and 99%, between 90 and 95%, between 95 and 99%, including any range or value therebetween. Each possibility represents a separate embodiment of the invention.

Non-limiting examples of suitable emulsifying and foam-forming agents include but are not limited to: non-ionic and anionic emulsifiers, such as polyoxyethylene-fatty acid esters, polyoxyethylene-fatty alcohol ethers, for example alkylaryl polyglycol ethers, alkylsulfonates, alkylsulfates, arylsulfonates as well as albumin hydrolyzation products.

Non-limiting examples of suitable dispersing agents include but are not limited to: lignin sulfite waste liquors and methylcellulose. Adhesives such as carboxymethyl cellulose and natural and synthetic polymers in the form of powders, granules or lattices, such as gum arabic, polyvinyl alcohol and polyvinyl acetate, as well as natural phospholipids, such as cephalins and lecithins, and synthetic phospholipids, can be used in the formulations. Further additives can be mineral and vegetable oils.

Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); organo-silicone surfactants include Silwet L77 (UAP), Silikin (Terra), Dyne-Amic (Helena), Kinetic (Helena), Sylgard 309 (Wilbur-Ellis) and Century (Precision); and polysorbate-type surfactants include Polysorbate 20 (Tween20), Polysorbate 40 (Tween40), Polysorbate 60 (Tween60), and Polysorbate 80 (Tween80).

In some embodiments, the surfactant is present at a concentration of between 0.01% v/v and 10% v/v. In some embodiments, the surfactant is present at a concentration of between 0.1% v/v and 1% v/v, between 0.1% v/v and 0.5% v/v, between 0.5% v/v and 1% v/v including any value therebetween. In some embodiments, the surfactant (e.g. Tween80) and an auxiliary solvent significantly enhance aqueous solubility of the compound.

In some embodiments, the composition comprises Tween80 at a concentration of between 0.01% v/v and 10% v/v, between 0.1% v/v and 1% v/v, between 0.1% v/v and 0.5% v/v, between 0.5% v/v and 1% v/v, including any range therebetween. In some embodiments, the composition comprises Tween20 at a concentration of between 0.01% v/v and 10% v/v, between 0.1% v/v and 1% v/v, between 0.1% v/v and 0.5% v/v, between 0.5% v/v and 1% v/v, including any range therebetween. Each possibility represents a separate embodiment of the invention.

Solid compositions can be prepared by dispersing the compound of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

In some embodiments, the agricultural composition is a field ready spray or a tank mix.

The compounds described hereinabove may be applied or otherwise utilized either as is, or as an agriculturally acceptable salt, enantiomer, diastereomer, solvate, or hydrate.

Non limiting examples of agriculturally acceptable salts include but are not limited to: cations derived from alkali or alkaline earth metals (e.g. sodium, potassium, magnesium), cations derived from ammonia and amines (e.g. ammonium, diethylammonium, ethanolammonium, isopropylammonium) and trimethylsulfonium salts.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

### Definitions

As used herein, the term “alkyl” describes an aliphatic hydrocarbon including straight chain and branched chain groups. Preferably, the alkyl group has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amino, hydroxyl, thiol, thioalkoxy, thiohydroxy, carboxy, amide, aryl and aryloxy, depending on the substituted group and its position in the molecule. Additional substituents are also contemplated.

The term “haloalkoxy” describes an alkoxy group as defined herein, further substituted by one or more halide(s).

The term “hydroxyl” or “hydroxy” describes a —OH group.

The term “mercapto” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amino” describes a —NR′R″ group, with R′ and R″ as described herein.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(O)OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” or “keto” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′ is as defined herein.

A “sulfinyl” group describes an —S(O)R′ group, where R′ is as defined herein.

A “sulfonyl” or “sulfonate” group describes an —S(O)₃R′ group, where R′ is as defined herein.

A “sulfone” group describes an —S(O)₂R′ group, where R′ is as defined herein.

A “carbamyl” or “carbamate” group describes an —OC(O)NR′R″ group, where R′ is as defined herein and R″ is as defined for R′.

A “nitro” group refers to a —NO₂ group.

The term “amide” as used herein encompasses C-amide and N-amide.

The term “C-amide” describes a —C(O)NR′R″ end group or a —C(O)NR′-linking group, as these phrases are defined hereinabove, where R′ and R″ are as defined herein.

The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N— linking group, as these phrases are defined hereinabove, with R′ as defined hereinabove.

The term “guanidine” describes a —R′NC(N)NR″R′″ end group or a —R′NC(N) NR″-linking group, as these phrases are defined hereinabove, where R′, R″ and R′″ are as defined herein.

As used herein, the term “azide” refers to a —N₃ group.

The term “sulfonamide” refers to a —S(O)₂NR′R″ group, with R′ and R″ as defined herein.

The term “phosphonyl” or “phosphonate” describes an —OP(O)—(OR′)₂ group, with R′ as defined hereinabove.

The term “phosphinyl” describes a —PR′R″ group, with R′ and R″ as defined hereinabove.

The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

The term “halide”, or “halo” describes fluorine, chlorine, bromine or iodine.

The term “haloalkyl” describes an alkyl group as defined above, further substituted by one or more halide(s).

The term “vinyl” refers to a —CH═CH₂ group.

The term “allyl” refers to a —CH₂—CH═CH₂ group.

The term “thioalkyl” describes an alkyl group as defined above, further substituted by one or more mercapto group(s).

The term “alkylhydroxy” describes an alkyl group as defined above, further substituted by one or more hydroxy group(s).

### General

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Synthetic Procedures

An exemplary synthetic procedure for preparation of candidate compounds is as follows:

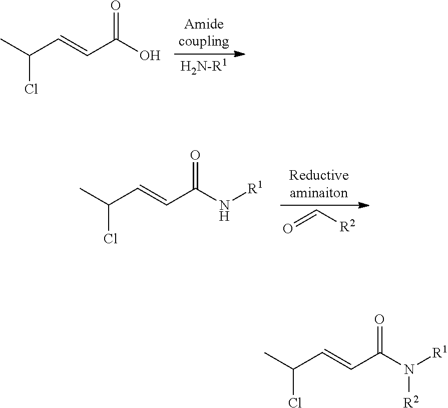

Compounds disclosed herein, can be synthesized based on the synthetic procedure described hereinabove, by using synthetic methods well-known in the art.

### 4-Chloro-2 pentenoic acid

To a mixture of NaCl (12 equiv.) and trioctylmethylammonium chloride (0.04 equiv.) in 100 ml of water was added a solution of 4-bromo-2-pentenoic acid (1 equiv.) in 4 ml of ethanol. After stirring for 2 hours at 80° C.−90° C., the reaction mixture was poured into water, acidified with diluted HCl, and extracted with ethyl acetate. The organic layer was washed with brine, dried over anhydrous sodium sulfate, filtrated and concentrated under reduced pressure. The residue was purified by distillation under reduced pressure to afford 4-chloro-2-pentenoic acid, as an oil.

### Amide Coupling

Amide coupling synthetic methods well-known art. Such reactions include the use of an amide coupling agent in the presence of an organic base (for example an aliphatic amine such as triethylamine or DIPEA) at room temperature. Amide coupling agents include, but are not limited to, BOP, PyBOP, PyBrOP, TBTU, TCTU, HBTU, HATU, T3P, COMU, TFFH, EDC, and DCC.

### Reductive Amination

Reductive amination reaction procedures are widely described in literature and are well known in the art. The amide intermediate can be reacted with (optionally substituted) aldehydes applying a reducing system such as, for example sodium borohydride, sodium triacetoxy-borohydride, sodium cyanoborohydride or di-n-butyltin dichloride with triphenyl-silane.

### Example 1

Paper Germination and Soil Germination

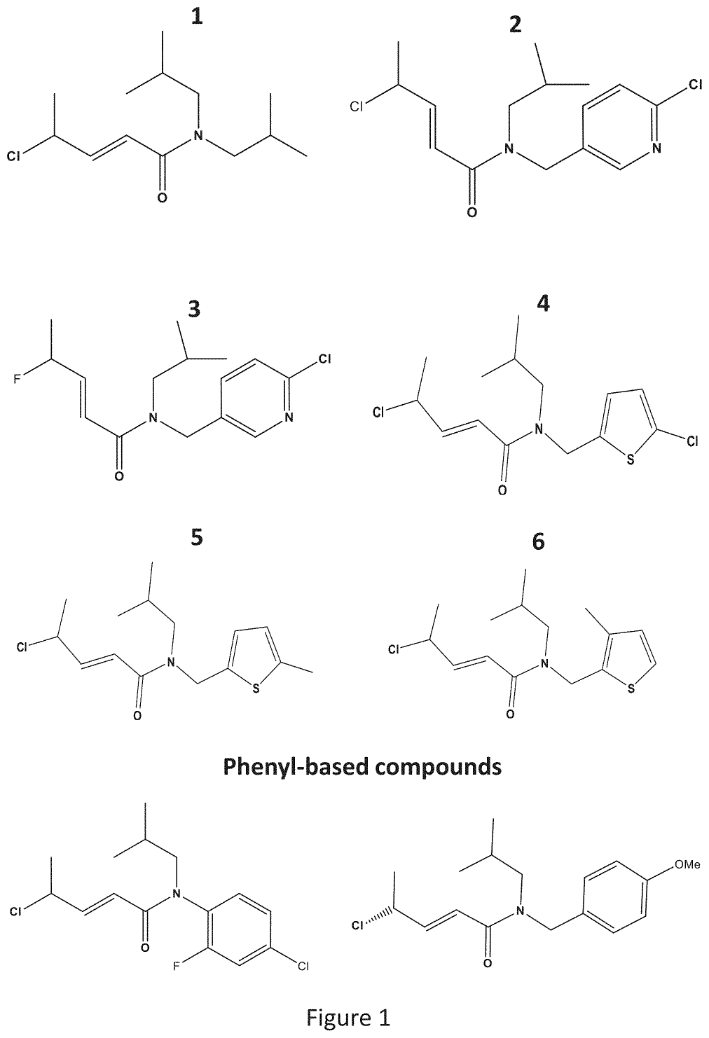

A series of candidate compounds 1-6, together with structurally similar phenyl-based derivatives (with chemical structures represented by FIG. 1), were evaluated for their herbicidal activity, as shown in Tables 1-7 hereinbelow.

### Paper Germination

### Materials

Compounds were dissolved in DMSO and then in distilled water to get 0.1 or 0.5 mg/ml solution with 0.5% DMSO.

0.5% DMSO solution in water was used as a negative control. As a positive control, 0.1 mg/ml oxyfluorfen (Galigan) solution was used.

### Experimental Procedure

A towel paper was placed in each box as bedding for seeds. Each box was soaked with 6 ml of the solutions on its paper. 13 seeds were placed on the wet paper and the box was sealed and incubated under conditions provided bellow.

Amaranthus palmeri—first 2 days under dark conditions. Plants were first incubated at 25° C. for 8 hr, then at 30° C. for 16 hr. Afterwards, plants were incubated at 25° C. for 8 hr in dark, followed by 16 hr incubation at 30° C. in light.

Lolium rigidum—first 4 days under dark conditions at 21° C. Afterwards, plants were incubated at 21° C. for 8 hr in dark, followed by 16 hr at 21° C. in light.

Gemination was assessed and scored after 3-7 days. Scoring scale: 0—complete germination inhibition, 100—no effect.

Compounds 4 and 5 significantly reduced germination of both Amaranthus palmeri and Lolium rigidum, as shown in Table 1. In contrary, fluorine and chlorine-substituted phenyl-based compound presented in FIG. 1 was not active with respect to paper germination reduction of Amaranthus palmeri and of Lolium rigidum at 0.1 mg/ml.

TABLE 1
Herbicidal activity evaluation for Amaranthus
palmeri and Lolium rigidum paper germination

| | Amaranthus palmeri | Lolium rigidum |
|---|---|---|
| Compound | paper germination | paper germination |
| No | 0.5 mg/ml | 0.5 mg/ml |
| 4 | 5 | 10 |
| 5 | 0 | 5 |

### Soil Germination

### Materials

Compounds were dissolved in DMSO and then in distilled water to get 0.01-2 mg/ml solution with 1% or 2% DMSO. 1% or 2% DMSO solution in water was used as a negative control. As a positive control, 0.1 or 0.2 mg/ml oxyfluorfen (Galigan) solution was used.

### Experimental Procedure

Flowerpots were filled with 30 g of soil, and 4-5 seeds were sowed in each flowerpot. Each treatment contained 3 flowerpots. Each flowerpot was sprayed with 1.5 ml of the dissolved compound. Soil was let dry for about 1 hour at room conditions, and then each flowerpot was sub-irrigated with 40 ml water and incubated under conditions provided below.

Amaranthus palmeri was incubated at 25° C. for 8 hr in dark, followed by 16 hr at 30° C. in light. Lolium rigidum was incubated at 21° C. for 8 hr in dark, followed by 16 hr at 21° C. in light. Germination was assessed and scored after 4-7 days. Scoring scale: 0—complete germination inhibition, 100—no effect.

Compounds 1 to 6 significantly reduced soil germination of Amaranthus palmeri, as shown in Table 2. In contrary, methoxy-substituted phenyl-based compound presented in FIG. 1 was substantially less active at 0.1 mg/ml. Furthermore, compounds 1, 4, 5 and 6 were active against both Amaranthus palmeri and Lolium rigidum. The fluorine and chlorine-substituted phenyl-based derivative and the methoxy-substituted phenyl-based derivative (FIG. 1) were unable to reduce soil germination of Lolium rigidum at 1 mg/ml and 0.5 mg/ml, respectively.

TABLE 2
Herbicidal activity evaluation for Amaranthus palmeri soil germination
Amaranthus palmeri soil germination

| Compound | 2 | 1 | 0.5 | 0.25 | 0.1 | 0.05 | 0.025 | 0.01 |
|---|---|---|---|---|---|---|---|---|
| No | mg/ml | mg/ml | mg/ml | mg/ml) | mg/ml | mg/ml | mg/ml | mg/ml |
| 1 | | | | 0 | 0 | 0 | 30 | 80 |
| 2 | | | | | 0 | 0 | 50 | 60 |
| 3 | | | | | 30 | | | |
| 4 | | 0 | 0 | 0 | 0 | | | |
| 5 | 0 | 0 | 30 | 70 | 100 | | | |
| 6 | 0 | 70 | 100 | 100 | 100 | | | |

TABLE 3
Herbicidal activity evaluation for Lolium rigidum
soil germination

| Compound | | |
|---|---|---|
| No | 2 mg/ml | 0.5 mg/ml |
| 1 | | 60 |
| 4 | 70 | 60 |
| 5 | 50 | 70 |
| 6 | 30 | |

### Example 2

Foliar Experiment

### Materials

Compounds were dissolved in DMSO and then in distilled water to get 0.1 or 0.5 mg/ml solution with 2% DMSO. Break-Thru surfactant was added to a final concentration of 0.05%. Negative control—an aqueous solution of 2% DMSO and 0.05% Break-Thru. Positive control—2 mg/ml Glyphosate (Roundup).

### Experimental Conditions

1.5 ml of each compound solution were sprayed on the foliage of different plants, which were 6-16 days old at the day of the experiment. Each treatment contained 3 pots. Plants were let dry for about 15 minutes at room conditions, and then incubated at controlled growth chambers. Phytotoxicity was assessed and scored 4-10 days after applications. Scoring scale: 0—complete phytotoxic effect, 100—no effect.

The experimental conditions were as follows (Table 4):

TABLE 4
Experimental conditions

| | No. of | Days | |
|---|---|---|---|
| | plants/ | after | |
| Plant | treatment | planting | Growth conditions |
| Amaranthus | 15 | 9 | 8 hr 25° C. dark/16 hr 30° C. |
| palmeri | | | light |
| Solarium | 12 | 12 | 8 hr 25° C. dark/16 hr 30° C. |
| nigrum | | | light |
| Abutilon | 12 | 12 | 8 hr 25° C. dark/16 hr 30° C. |
| theophrasti | | | light |
| Lactuca | 18 | 14 | 8 hr 25° C. dark/16 hr 30° C. |
| sativa | | | light |
| Sinapis alba | 15 | 13 | 8 hr 21° C. dark/16 hr 21° C. |
| | | | light |
| Gossypium | 12 | 13 | 8 hr 25° C. dark/16 hr 30° C. |
| hirsutum | | | light |
| Brassica | 15 | 13 | 8 hr 21° C. dark/16 hr 21° C. |
| napus | | | light |
| Echinochloa | 30 | 12 | 8 hr 25° C. dark/16 hr 30° C. |
| colonum | | | light |
| Setaria | 15 | 9 | 8 hr 25° C. dark/16 hr 30° C. |
| adhaerens | | | light |
| Lolium | 15 | 8 | 8 hr 21° C. dark/16 hr 21° C. |
| rigidum | | | light |
| Phalaris | 90 | 8 | 8 hr 21° C. dark/16 hr 21° C. |
| brachystachys | | | light |
| Zea mays | 12 | 6 | 8 hr 25° C. dark/16 hr 30° C. |
| | | | light |
| Triticum | 15 | 16 | 8 hr 21° C. dark/16 hr 21° C. |
| vulgare | | | light |

As shown in Table 5, compounds 4 and 5 showed solid herbicidal activity in this experiment, in a variety of plants. Compound 2 also showed solid herbicidal activity against Amaranthus palmeri.

All tested compounds were ineffective against Zea mays and Triticum vulgare.

TABLE 5
Herbicidal activity evaluation

| | Compound No | | |
|---|---|---|---|
| | Concentration (mg/ml) | | |
| | 5 | 4 | 2 |
| | 0.5 | 0.5 | 0.1 |
| Plant | (mg/ml) | (mg/ml) | (mg/ml) |
| Amaranthus | 30 | 0 | 0 |
| palmeri | | | |
| Solarium | 40 | 0 | |
| nigrum | | | |
| Abutilon | 20 | 5 | |
| theophrasti | | | |
| Lactuca | 0 | 0 | |
| sativa | | | |
| Sinapis alba | 30 | 10 | |
| Gossypium | 40 | 60 | |
| hirsutum | | | |
| Brassica | 0 | 0 | |
| napus | | | |
| Echinochloa | 70 | 70 | |
| colonum | | | |
| Setaria | 10 | 5 | |
| adhaerens | | | |
| Lolium | 30 | 10 | |
| rigidum | | | |
| Phalaris | 40 | 5 | |
| brachystachys | | | |
| Zea mays | 90 | 80 | 90 |
| Triticum | 90 | 100 | 90 |
| vulgare | | | |

### Example 3

Soil Germination

### Materials

Compounds were dissolved in DMSO and then in distilled water to get 0.1 or 0.5 mg/ml solution with 2% DMSO. 2% DMSO solution in water was used as a negative control. No positive control was used in this assay.

### Experimental Conditions

Flowerpots were filled with 30 g of soil, each treatment contained three flowerpots. Each flowerpot was sprayed with 1.5 ml of the dissolved compound. Soil was let dry for about 1 hour at room conditions, and then each flowerpot was sub-irrigated with 40 ml water and incubated at controlled growth chambers, under conditions provided on table 6.

Gemination was assessed and scored after 7 days. Scoring scale: 0—complete germination inhibition, 100—no effect.

TABLE 6
Experiment conditions

| | No. of | |
|---|---|---|
| | seeds/ | |
| Plant | treatment | Growth conditions |
| Amaranthus palmeri | 15 | 8 hr 25° C. dark/16 hr 30° C. light |
| Solarium nigrum | 12 | 8 hr 25° C. dark/16 hr 30° C. light |
| Abutilon theophrasti | 12 | 8 hr 25° C. dark/16 hr 30° C. light |
| Lactuca sativa | 18 | 8 hr 25° C. dark/16 hr 30° C. light |
| Sinapis alba | 15 | 8 hr 21° C. dark/16 hr 21° C. light |
| Gossypium hirsutum | 12 | 8 hr 25° C. dark/16 hr 30° C. light |
| Brassica napus | 15 | 8 hr 21° C. dark/16 hr 21° C. light |
| Echinochloa | 30 | 8 hr 25° C. dark/16 hr 30° C. light |
| colonum | | |
| Setaria adhaerens | 15 | 8 hr 25° C. dark/16 hr 30° C. light |
| Lolium rigidum | 15 | 8 hr 21° C. dark/16 hr 21° C. light |
| Zea mays | 12 | 8 hr 25° C. dark/16 hr 30° C. light |
| Triticum vulgare | 15 | 8 hr 21° C. dark/16 hr 21° C. light |

Alkyl-based compound 1, pyridine-based compound 2 and thiophene-based compounds 4 and 5 were tested in various plant growth experiments as shown in Table 7.

TABLE 7
Herbicidal activity evaluation

| | Compound No | | | |
|---|---|---|---|---|
| | Concentration (mg/ml) | | | |
| | 4 | 5 | 1 | 2 |
| Plant | 0.5 mg/ml | 0.5 mg/ml | 0.1 mg/ml | 0.1 mg/ml |
| Amaranthus | 0 | 0 | 0 | 0 |
| palmeri | | | | |
| Solarium nigrum | 10 | 100 | | |
| Abutilon | 10 | 80 | 100 | 100 |
| theophrasti | | | | |
| Lactuca sativa | 70 | 70 | | |
| Sinapis alba | 30 | 50 | | |
| Gossypium | 90 | 100 | | |
| hirsutum | | | | |
| Brassica napus | 10 | 20 | | |
| Echinochloa | 5 | 100 | 100 | 100 |
| colonum | | | | |
| Setaria adhaerens | 5 | 10 | 60 | 80 |
| Lolium rigidum | 60 | 70 | | 90 |
| Zea mays | 80 | 90 | 100 | 100 |
| Triticum vulgare | 60 | 100 | 100 | 100 |

### Example 4

Contact Experiment

### Materials

Compounds were dissolved in DMSO and then in distilled water to get 0.25 or 0.5 mg/ml solution with 2% DMSO. Break-Thru surfactant was added to a final concentration of 0.05%.

Negative control—aqueous solution of 2% DMSO and 0.05% Break-Thru. No positive control was used in this assay.

### Experimental Conditions

Compounds were applied on the foliage of 13 days old Amaranthus palmeri plants. Each treatment contained 3 to 4 plants, where on each plant 3 leaves were treated. Each leaf was treated with a total of 10-40 μl which was applied as droplets at 10-15 spots on the leaf surface. Plants were grown at controlled growth chambers (8 hr 25° C. dark/16 hr 30° C. light) and were assessed 2-5 day after applications.

Compounds tested in this assay: compound 1, compound 2, and compound 3.

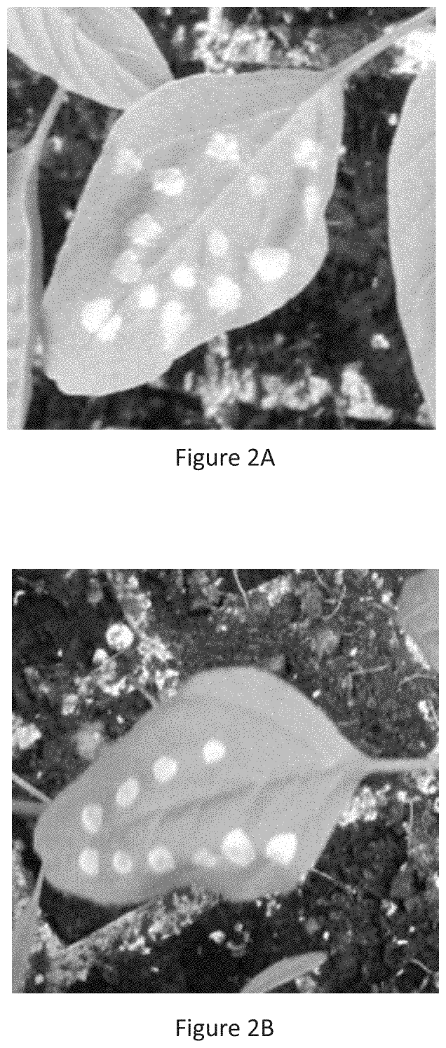

As demonstrated in FIGS. 2A-B, all compounds showed damage at the point of contact, with no apparent damage to other parts of the plant, suggesting that these compounds act as contact herbicides.

### Example 5

Pre-Emergence and Post Emergence Application

### Materials

For pre-emergence application, compounds were dissolved in DMSO and then in distilled water to get 0.1-2 mg/ml solution with 2% DMSO.

2% DMSO solution in water was used as a negative control. As a positive control, 0.2 mg/ml oxyfluorfen (Galigan) solution was used.

For post-emergence application, compounds were dissolved in DMSO and then in distilled water to get 0.05-2 mg/ml solution with 2% DMSO. Break-Thru surfactant was added to a final concentration of 0.05%.

Negative control—an aqueous solution of 2% DMSO and 0.05% Break-Thru. Positive control—2 mg/ml Glyphosate (Roundup).

### Experimental Conditions

For pre-emergence application, flowerpots were filled with 30 g of soil, and 4-5 Amaranthus palmeri seeds were sowed in each flowerpot. Each treatment contained 3 flowerpots. Each flowerpot was sprayed with 1.5 ml of the dissolved compound. Soil was let dry for about 1 hour at room conditions, and then each flowerpot was sub-irrigated with 40 ml water and incubated under conditions provided below.

Amaranthus palmeri was incubated at 25° C. for 8 hr in dark, followed by 16 hr at 30° C. in light. Germination was assessed and scored after 4-7 days. Scoring scale: 0—complete germination inhibition, 100—no effect.

For post-emergence application, 1.5 ml of each compound solution were sprayed on the foliage of Amaranthus palmeri, which were 10 days old at the day of the experiment. Each treatment contained 3 pots. Plants were let dry for about 15 minutes at room conditions, and then incubated at controlled growth chambers. Phytotoxicity was assessed and scored 4-10 days after applications. Scoring scale: 0—complete phytotoxic effect, 100—no effect.

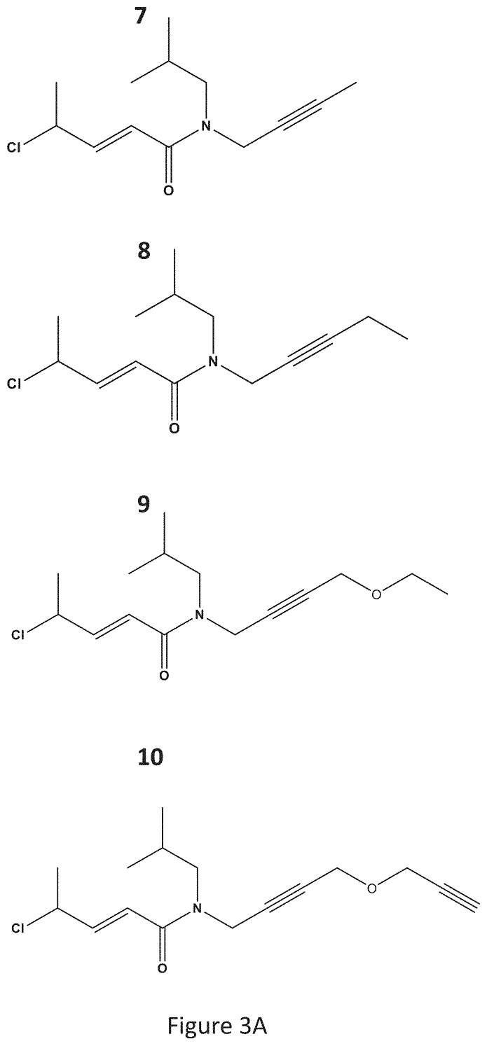

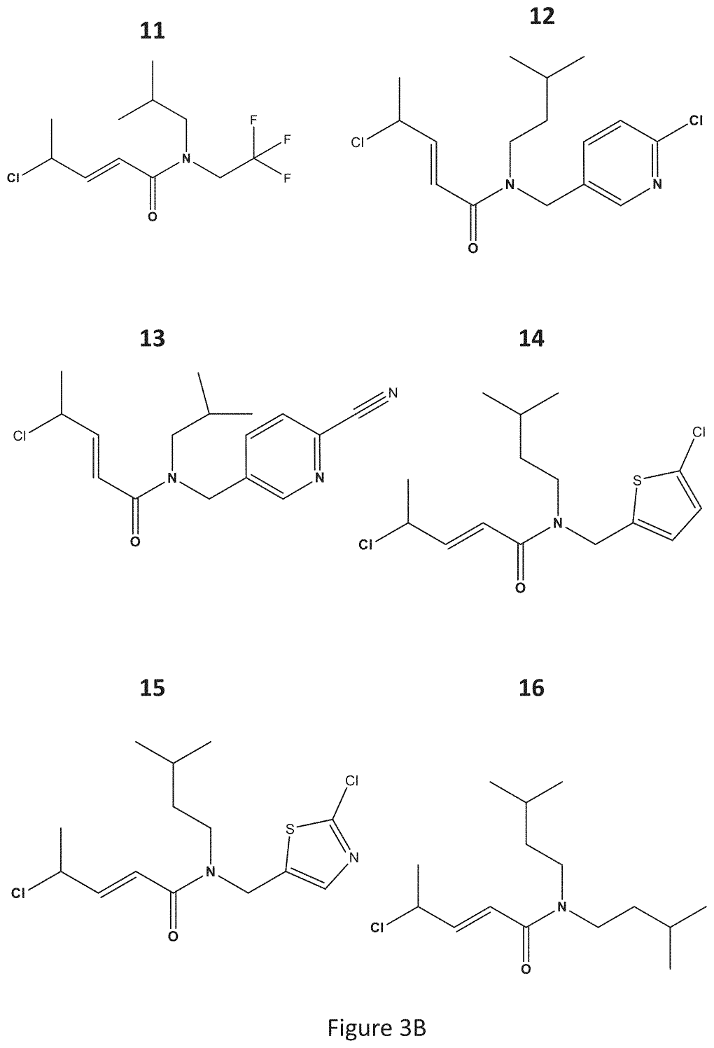

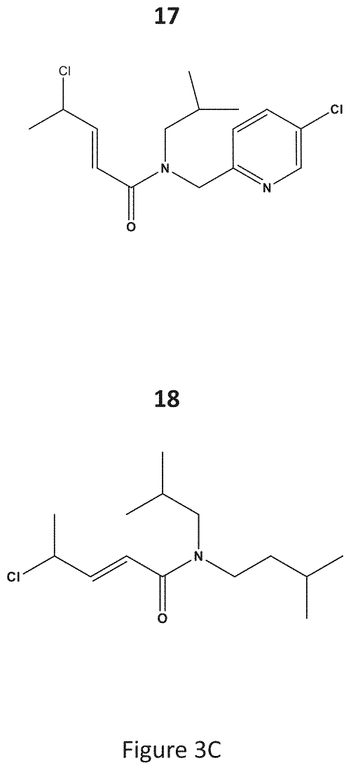

A series of candidate compounds 7-18, (with chemical structures represented by FIGS. 3A-C), were evaluated for their herbicidal activity, as shown in Tables 8-9 hereinbelow.

TABLE 8
Pre-emergence Amaranthus palmeri
herbicidal activity evaluation

| Compound | Concentration (mg/ml) | | |
|---|---|---|---|
| No | 2 | 0.25 | 0.1 |
| 7 | 0 | 5 | 10 |
| 8 | 0 | 40 | 100 |
| 9 | 0 | 50 | 60 |
| 10 | 0 | 5 | 100 |
| 11 | 0 | 5 | 15 |
| 12 | | 5 | 20 |
| 13 | | 0 | 30 |
| 14 | | 5 | 20 |
| 15 | | 0 | 20 |
| 16 | | 0 | 10 |

TABLE 9
Post-emergence Amaranthus palmeri
herbicidal activity evaluation

| Compound | Concentration (mg/ml) | | | | |
|---|---|---|---|---|---|
| No | 2 | 0.5 | 0.25 | 0.075 | 0.05 |
| 7 | 40 | | | | |
| 8 | 30 | | | | |
| 9 | 80 | | | | |
| 10 | 70 | | | | |
| 11 | 60 | | | | |
| 12 | | | | 0 | 0 |
| 13 | | | | 0 | 10 |
| 14 | | 20 | 20 | | |
| 15 | | 0 | 5 | | |
| 16 | | 10 | 50 | | |

Compounds 7-16 presented an herbicidal effect against Amaranthus palmeri upon applying to a plant, or the area under cultivation.

Compounds 12, 13, 14 and 15 presented moderate herbicidal effect against Setaria viridis. Compound 16 presented a very good herbicidal effect (more than 30% inhibition) against Setaria viridis upon applying to the area under cultivation.

The study of the herbicidal effect of compounds 17 and 18 against Amaranthus palmeri and Setaria viridis upon applying to a plant, or the area under cultivation, are currently ongoing.

### Example 6

Herbicidal Compositions

Compounds 1 and 2 were used in exemplary herbicidal formulations as described hereinbelow.

### Formulation I:

Compound 1 or 2 at a concentration ranging from 0.5 mg/ml to 5 mg/ml.

For compound 1 or 2 at a concentration of 5 mg/ml:

4% DMSO

0.5% Tween80

0.05% Break-Thru (for post-emergence application)

### Formulation II:

Compound 1 or 2 at a concentration ranging from 5 mg/ml to 10 mg/ml.

For compound 1 or 2 at a concentration of 10 mg/ml:

3% Xylene

0.4% emulsifiers

0.05% Break-Thru (for post-emergence application)

The emulsifiers used were Ethoxylated Castor Oil-(Kolliphor® RH 40, Manufactured by Sigma-Aldrich) (ECO) and Ca-LAS-Linear Alkylbenzene Sulfonate (Rhodacal® 60 be, Manufactured by Solvay) (Ca-LAS).

For compound 1, different ratios of ECO and Ca-LAS were tested: 50:50, 60:40, 80:20, and 90:10. The 90:10 ratio was used for net-house and green-house experiments.

For compound 2 ECO and Ca-LAS were used in a ratio of 60:40.

### Formulation III:

Table 10 presents the content of compounds used and their percentage in the formulation and in the spraying solution after addition of H₂O.

TABLE 10
Formulation III

| | Compound 2 | Xylene | ECO | Ca-LAS | H₂O |
|---|---|---|---|---|---|
| % of | 22.7% | 68.2% | 5.5% | 3.6% | — |
| formulation | | | | | |
| % in | 1% | 3% | 0.24% | 0.16% | 95.6% |
| spraying | | | | | |
| solution | | | | | |
| Weight in | 1 g | 3 g | 240 mg | 160 mg | 95.6 g |
| spraying | | | | | |
| solution | | | | | |

### Example 7

Nethouse and Greenhouse Assays

Herbicidal activity of Compounds 1, 4 and 5 was further examined under nethouse and greenhouse conditions.

All compound showed good herbicidal activity at pre- and post-emergence application on various types of weeds, including Amaranthus palmeri, Echinochloa colonum, Solanum nigrum, Setaria viridis and Lolium multiflorum.

Notably, germination and growth of Triticum vulgare, Gossypium hirsutum and Zea mays were not affected by the application rates used in those assays.

### Example 8

Residual Activity Assays

Residual activity of Compound 1 and Compound 2 was examined in greenhouse assay. Three weeks after pre-emergence application, new Amaranthus palmeri seeds were sowed on the same pots and germination was followed. While germination was observed in the control and Compound 1 treatments, no germination was observed in pots sprayed with Compound 2.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-49. (canceled)

50. A herbicidal composition comprising an effective amount of a compound, or a salt thereof, wherein said compound is represented by Formula IIIc: 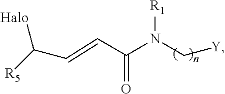
wherein:
Y represents an isopropyl group, (C₂-C₁₀) alkyl group, a substituted (C₂-C₁₀) alkyl group, an alkyne group, a substituted alkyne group, a (C₃-C₁₀) cycloalkyl group, a substituted (C₃-C₁₀) cycloalkyl, a heteroaryl, a substituted heteroaryl, a heterocyclyl, a substituted heterocyclyl, an aliphatic ring, a bicyclic aliphatic ring, or a combination thereof;
R₁ is selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an alkylhydroxy group, (C₁-C₅ alkyl)-R₂—(C₁-C₅ alkyl)₀₋₂, an alkoxy group, an amino group, a guanidine group, a thioalkoxy group, a thioalkyl group, a hydroxy group, a mercapto group, an allyl group, a vinyl group, a cyano group, a nitro group, an alkylamino group, an alkylamide group, a keto group, a halo group, a haloalkyl group, an azo group and a carboxylic acid derivative;
R₅ is selected from the group consisting of: a linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a halo group, a C₁-C₇ alkoxy group, an amino group, a thioalkoxy group, a thioalkyl group, a hydroxy group, a mercapto group, an allyl group, C₁-C₇ ether group, a vinyl group, a cyano group, a nitro group, a C₁-C₇ alkylamino group and a C₁-C₇ alkylamide group or any combination thereof; and
n represents an integer in a range from 0 to 10.

51. The herbicidal composition of claim 50, wherein said compound is represented by Formula IIId: 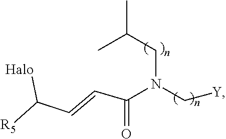
wherein each n independently represents an integer in a range from 0 to 5.

52. The herbicidal composition of claim 50, wherein Y represents an isopropyl group, (C₂-C₁₀) alkyl group, a substituted (C₂-C₁₀) alkyl group, an alkyne group, a substituted alkyne group, a trifluoromethyl group, a pyran, a thiopyran, a pyridine, a substituted pyran, a substituted thiopyran, a substituted pyridine, a thiophene, a substituted thiophene, a thiazole, a substituted thiazole, an imidazole, a substituted imidazole, a furan, a substituted furan, or a combination thereof.

53. The herbicidal composition of claim 50, wherein R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

54. The herbicidal composition of claim 51, wherein Y represents an isopropyl group, (C₂-C₁₀) alkyl group, a substituted (C₂-C₁₀) alkyl group, an alkyne group, a substituted alkyne group, a trifluoromethyl group, a pyran, a thiopyran, a pyridine, a substituted pyran, a substituted thiopyran, a substituted pyridine, a thiophene, a substituted thiophene, a thiazole, a substituted thiazole, an imidazole, a substituted imidazole, a furan, a substituted furan, or a combination thereof.

55. The herbicidal composition of claim 51, wherein R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

56. The herbicidal composition of claim 54, wherein R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

57. The herbicidal composition of claim 50, wherein said compound is represented by Formula IVd: 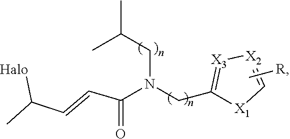
wherein:
X₁, X₂, and X₃ are independently selected from the group consisting of: C, CH, S, O, N, and NH; and
R represents a substituent, independently selected from the group consisting of: a halo group, an alkyl group, hydrogen, an alkoxy group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

58. The herbicidal composition of claim 50, wherein said compound is represented by Formula VIIa: 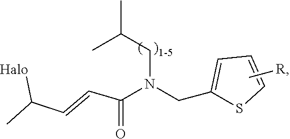
or by Formula VIIb:

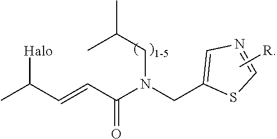

59. The herbicidal composition of claim 58, wherein said compound is selected from the group consisting of:

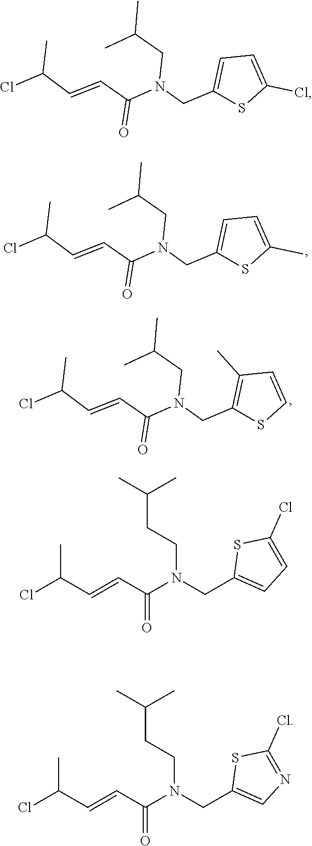

60. The herbicidal composition of claim 50, wherein said compound is represented by Formula IXc:

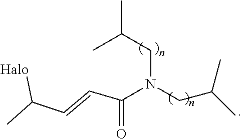

61. The herbicidal composition of claim 59, wherein said compound is selected from the group consisting of:

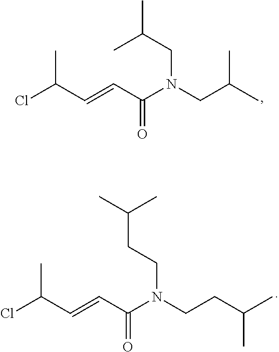 [SMILES: CC(Cl)/C=C/C(=O)N(CC(C)C)CC(C)C ; CC(Cl)/C=C/C(=O)N(CCC(C)C)CCC(C)C]

62. The herbicidal composition of claim 50, wherein said compound is represented by Formula IXd:

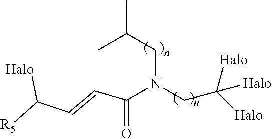

63. The herbicidal composition of claim 50, wherein said compound is represented by Formula XIc: 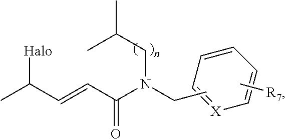
wherein:
X is N;
n represents an integer in a range from 0 to 5; and
R₇ represents a substituent, selected from the group consisting of: a halo group, an alkyl group, an alkoxy group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

64. The herbicidal composition of claim 63, wherein said compound is represented by Formula XIIc:

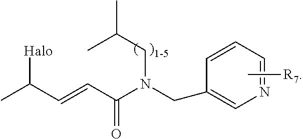

65. The herbicidal composition of claim 64, wherein said compound is selected from the group consisting of:

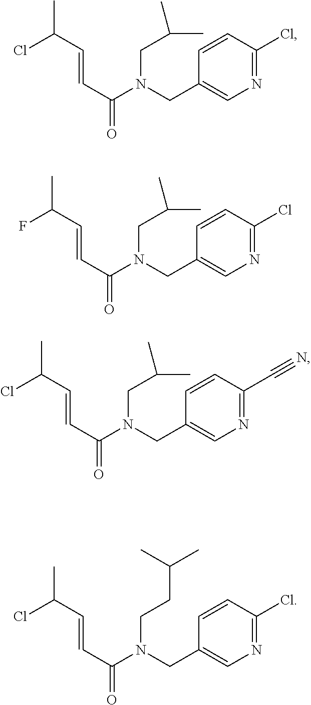 [SMILES: CC(Cl)/C=C/C(=O)N(Cc1ccc(Cl)nc1)CC(C)C ; CC(F)/C=C/C(=O)N(Cc1ccc(Cl)nc1)CC(C)C ; CC(Cl)/C=C/C(=O)N(Cc1ccc(C#N)nc1)CC(C)C ; CC(Cl)/C=C/C(=O)N(CCC(C)C)Cc1ccc(Cl)nc1]

66. The herbicidal composition of claim 50, wherein said compound is represented by Formula XVa: 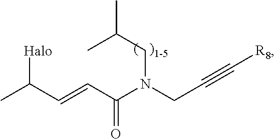
wherein:
R₈ represents a substituent, selected from the group consisting of: methyl group, isopropyl group, an alkylhydroxy group, an alkoxy group, a halo group, an alkyl group, a substituted alkyl group, an alkyne group, an amino group, a guanidine group, a thioalkoxy group, a hydroxy group, a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, a hydrogen, or any combination thereof.

67. The herbicidal composition of claim 66, wherein said compound is selected from the group consisting of:

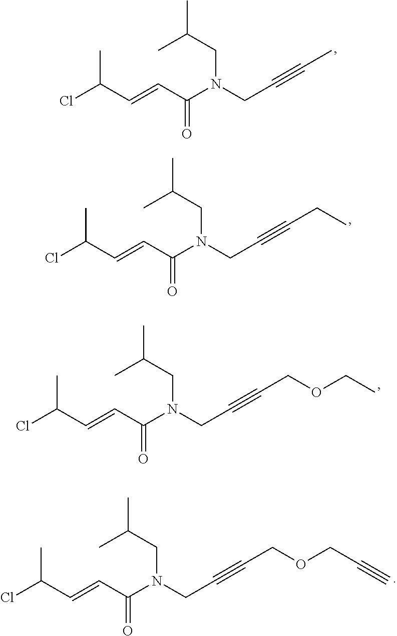

68. The herbicidal composition of claim 50, comprising an additive selected from a solvent, a surfactant, an emulsifier, or any combination thereof.

69. The herbicidal composition of claim 50, comprising 5% to 40% weight per weight (w/w) of said compound.

70. The herbicidal composition of claim 50, further comprising an agriculturally acceptable carrier.

71. A compound, or a salt thereof, wherein said compound is represented by Formula IIIc: 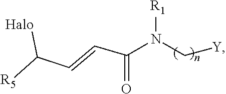
wherein:
Y represents an isopropyl group, (C₂-C₁₀) alkyl group, a substituted (C₂-C₁₀) alkyl group, an alkyne group, a substituted alkyne group, a (C₃-C₁₀) cycloalkyl group, a substituted (C₃-C₁₀) cycloalkyl, a heteroaryl, a substituted heteroaryl, a heterocyclyl, a substituted heterocyclyl, an aliphatic ring, a bicyclic aliphatic ring, or a combination thereof;
R₁ is selected from the group consisting of: methyl group, isopropyl group, (C₂-C₁₀) alkyl group, a substituted (C₂-C₁₀) alkyl group, a (C₃-C₁₀) cycloalkyl group, an alkyne group, a substituted alkyne group, an alkylhydroxy group, (C₁-C₅ alkyl)-R₂—(C₁-C₅ alkyl)₀₋₂, an alkoxy group, an amino group, a guanidine group, a thioalkoxy group, a thioalkyl group, a hydroxy group, a mercapto group, an allyl group, a vinyl group, a cyano group, a nitro group, an alkylamino group, an alkylamide group, a keto group, a halo group, a haloalkyl group, an azo group and a carboxylic acid derivative;
R₅ is selected from the group consisting of: a linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a halo group, a C₁-C₇ alkoxy group, an amino group, a thioalkoxy group, a thioalkyl group, a hydroxy group, a mercapto group, an allyl group, C₁-C₇ ether group, a vinyl group, a cyano group, a nitro group, a C₁-C₇ alkylamino group and a C₁-C₇ alkylamide group or any combination thereof; and
n represents an integer in a range from 0 to 10.

72. The compound of claim 71, wherein said compound is represented by Formula IIId: 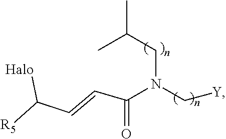
Wherein each n independently represents an integer in a range from 0 to 5.

73. The compound of claim 71, wherein Y represents an isopropyl group, (C₂-C₁₀) alkyl group, a substituted (C₂-C₁₀) alkyl group, an alkyne group, a substituted alkyne group, a trifluoromethyl group, a pyran, a thiopyran, a pyridine, a substituted pyran, a substituted thiopyran, a substituted pyridine, a thiophene, a substituted thiophene, a thiazole, a substituted thiazole, an imidazole, a substituted imidazole, a furan, a substituted furan, or a combination thereof.

74. The compound of claim 71, wherein R₈ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

75. The compound of claim 72, wherein Y represents an isopropyl group, (C₂-C₁₀) alkyl group, a substituted (C₂-C₁₀) alkyl group, an alkyne group, a substituted alkyne group, a trifluoromethyl group, a pyran, a thiopyran, a pyridine, a substituted pyran, a substituted thiopyran, a substituted pyridine, a thiophene, a substituted thiophene, a thiazole, a substituted thiazole, an imidazole, a substituted imidazole, a furan, a substituted furan, or a combination thereof.

76. The compound of claim 72, wherein R₈ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

77. The compound of claim 75, wherein R₅ is selected from the group consisting of a linear C₁-C₅ alkyl group, a branched C₁-C₅ alkyl group, a C₁-C₇ haloalkyl group, C₁-C₅ ether group, a C₁-C₇ alkylhydroxy group, and a halo group or any combination thereof.

78. A method for controlling plant growth, comprising applying to the plant, a part of the plant, a seed of the plant, or the area under cultivation, an effective amount of the herbicidal composition of claim 50.

79. The method of claim 78, wherein said plant is selected from a crop plant and a weed.

80. The method of claim 78, wherein said effective amount is between 0.5 grams per hectare (g/ha) and 2000 g/ha.
